(19) United States
(12) Patent Application Publication
(10) Pub. No.: US 2017/0348174 A1
(43) Pub. Date: Dec. 7, 2017

(54) METHOD AND SYSTEM FOR PHYSICAL STIMULATION OF TISSUE

(71) Applicants: University of Illinois, Urbana, IL (US); The Research Foundation for the State University of New York, Albany, NY (US)

(72) Inventors: Clinton Rubin, Port Jefferson, NY (US); Stefan Judex, Port Jefferson, NY (US); Timothy J. Koh, Wheaton, IL (US); Giamila Fantuzzi, Chicago, IL (US)

(21) Appl. No.: 15/683,458

(22) Filed: Aug. 22, 2017

Related U.S. Application Data
(63) Continuation of application No. 15/273,240, filed on Sep. 22, 2016, now abandoned.
Application No. 15/273,240 is a continuation of application No. 14/969,636, filed on Dec. 15, 2015, now abandoned.
Application No. 14/969,636 is a continuation of application No. 13/768,710, filed on Feb. 15, 2013, now abandoned.
Application No. 13/768,710 is a continuation-in-part of application No. 12/300,958, filed on Sep. 3, 2009, now abandoned.
Application No. 13/768,710 is a continuation-in-part of application No. 12/919,533, filed on Dec. 1, 2010, now abandoned.
(60) Provisional application No. 60/801,325, filed on May 17, 2006.
(60) Provisional application No. 61/032,942, filed on Feb. 29, 2008.

(51) Int. Cl. A61H 1/00; A61N 2/02; A63B 21/00; A61H 23/02; A61H 33/00

(52) U.S. Cl. CPC A61H 1/00; A63B 21/00196; A61H 1/005; A61H 23/02; A61H 23/0218; A61H 23/0245; A61N 2/02; A61H 2205/083; A63B 2213/004; A61H 2203/0443; A61H 2203/0425; A61H 2203/0412; A61H 2203/0406; A61H 2203/03; A61H 2201/164; A61H 2201/1628; A61H 2201/1623; A61H 2201/0157; A61H 2201/0153; A61H 2205/108; A61H 2230/30; A61H 2230/70; A61H 2033/0029; A63B 21/4037; A61H 2205/081

(57) ABSTRACT

Methods and systems of applying physical stimuli to tissue are disclosed. The methods can include reducing or suppressing pancreatitis in a subject by administering a low magnitude, high frequency mechanical signal on a period basis and for a time sufficient to reduce or suppress pancreatitis. The methods can include enhancing healing of damaged tissue in a subject by administering to the subject a low magnitude, high frequency mechanical signal on a periodic basis and for a time sufficient to treat the damaged tissue. The systems can include a device for generating a low magnitude, high frequency physical signal and a platform for applying the low magnitude, high frequency physical signal to the subject for a predetermined time.

### CROSS-REFERENCE TO RELATED APPLICATIONS

This application claims priority to, and the benefit of, U.S. patent application Ser. No. 12/300,958, filed on Nov. 14, 2008, which is a National Stage application under 35 U.S.C. §371 and claims the benefit under 35 U.S.C. §119(a) of International Application No. PCT/US07/69154, filed on May 17, 2007, which claims the benefit of priority under 35 U.S.C. §119(e) to U.S. Application Ser. No. 60/801,325, filed on May 17, 2006, and U.S. patent application Ser. No. 12/919,533, filed on Aug. 26, 2010, which is a National Stage application under 35 U.S.C. §371 and claims the benefit under 35 U.S.C. §119(a) of International Application No. PCT/US09/35777, filed on Mar. 2, 2009, which claims the benefit of priority under 35 U.S.C. §119(e) to U.S. Application Ser. No. 61/032,942, which was filed on Feb. 29, 2008, all of which are incorporated herein by reference in their entirety.

### FEDERALLY SPONSORED RESEARCH OR DEVELOPMENT

The U.S. government may have certain rights in this invention pursuant to Grant Nos. AR 43498, AR 52778, DK083328, and GM092850 awarded by the National Institutes of Health and Grant No. NAG 9-1499 awarded by the National Aeronautics and Space Administration.

### TECHNICAL FIELD

This disclosure describes a treatment for weight control or weight gain and for related conditions, such as diabetes, that is non-invasive and non-pharmaceutical. More particularly, we describe an intervention in which low level, high frequency mechanical signals are applied to subjects for the suppression of weight gain and for the treatment or prevention of other undesirable conditions. As a result of improved weight control and/or by independent means, the present treatments can maintain or improve insulin resistant states and inhibit conditions associated with obesity, such as cardiovascular disease and hypertension.

This disclosure also relates to methods for altering the differentiation and proliferation of cells, including stem cells, in cell culture or in patients who have had, for example, a traumatic injury. The methods can also be used, for example, to counteract a side effect of chemotherapy or radiation therapy or to improve the outcome of a transplant, such as a bone marrow transplant.

### BACKGROUND

Obesity and diabetes are prevalent in the United States and are becoming more prevalent in other countries. In the U.S. alone, these conditions affect millions of people and encumber billions in annual health care service costs. Despite significant public attention, effective pharmacologic interventions at any scale have proven elusive. Even control of obesity and diabetes has proven difficult, with perhaps the only common etiologic factor being “a sedentary lifestyle,” and the only common intervention being exercise. The need for new treatment and prevention strategies is apparent.

New treatment strategies are also needed for healing injured or damaged tissue. Healing may be delayed in cases of trauma-induced injuries, as well as in chronic wounds that fail to progress through the usual phases of healing. Common treatments to aid in healing of traumatic tissue injuries and chronic wounds have been shown to be ineffective in restoring the structure and function of tissue. Exploratory treatments utilizing stem cells, growth factors, and anti-fibrotic drugs have shown promise in improving healing in animal models, but are often complicated, expensive, and may induce serious side effects.

### SUMMARY

According to one aspect of the present disclosure, the information that follows is based, in part, on our discovery that applying brief periods of low-magnitude, high-frequency mechanical signals to a subject (e.g., on a daily basis) can suppress adipogenesis, improve the subject's metabolic state (e.g., by markedly reducing free fatty acids and/or triglycerides in liver, muscle and/or adipose tissue), and improve glucose tolerance. While the present methods are not limited to those that produce a particular cellular response, our data indicate that the benefits we have observed are not achieved by elevating the subject's metabolism, as might occur with exercise, but primarily by suppressing the differentiation of precursor cells into adipocytes, thus biasing progenitors against a commitment to fat and inhibiting the etiologic progression of certain diseases, including those directly pronounced by obesity.

Accordingly, the invention features methods of altering (e.g., reducing) a subject's weight or promoting the maintenance of a healthier weight; of reducing or suppressing the further accumulation of subcutaneous fat; of reducing or inhibiting the further incorporation of fat in muscle or internal organs; of reducing or suppressing the further accumulation of visceral fat around internal organs; and/or of inhibiting the development or progression of obesity and disorders correlated with either excess weight per se or an undesirable fat distribution (e.g., fat accumulation around internal organs). These outcomes can occur in the course of maintaining or improving a subject's metabolic state, which is discussed in more detail below. Regardless of whether the methods are described with respect to a particular physiological parameter (such as a subject's weight) or more generally as being applicable to metabolic state or to a suspected or diagnosed condition (e.g., diabetes), the methods can be carried out by providing to the subject a low-magnitude, high-frequency physical signal. For example, the signal can be supplied to reduce the amount of visceral or subcutaneous fat or to suppress the rate of its production. The signal can also be supplied to maintain or improve the subject's metabolic state as evidenced, for example, by the rate of carbohydrate metabolism or lipid metabolism. Because our data indicate these physical signals can influence the fate of mesenchymal stem cells, the present methods can also be used to help retain or restore bone marrow viability and to direct the controlled differentiation of stem cells, including those placed in cell culture, down specific pathways. Our data further indicate that the physical signals described herein can upregulate peroxisome proliferative activated receptors gamma (PPAR-γ) and downregulate arachidonate 15-lipoxygenase (Alox15), both of which are associated with lipid metabolism. The upregulation of PPAR-γ and/or the downregulation of Alox15 can therefore be used to assess the adequacy of a given physical signal, as can non-molecular level indicators such as weight, fat distribution, and BMI, and such evaluation methods are within the scope of the present invention. Where molecular level indicators, including those discussed here or others that indicate cellular differentiation, are assessed, one may do so in vitro or in cell culture. Expression levels may be assessed in samples (e.g., blood, fat, urine, or bone marrow samples) obtained from animals serving as animal models or from human patients.

As noted above, the present methods encompass those for maintaining or improving the metabolic state of a subject (e.g., a human of any age; children, adolescents, and adults, including the elderly, can all be treated). The methods can, optionally, include a step by which one identifies a suitable subject and a step of providing to the subject a low magnitude, high frequency mechanical signal on a periodic basis and for a time sufficient to maintain or improve the subject's metabolic state. Where the optional identification step is included, one can evaluate a physiological parameter that reflects the metabolic state of the subject. The parameter can be, for example, the level, in the subject (e.g., a level in the subject's blood or urine) of: a triglyceride, a free fatty acid, a cholesterol, fibrinogen, C-reactive protein, hemoglobin Alc, insulin, glucose, a pro-inflammatory cytokine, or an adipokine. Other parameters, any of which can be assessed either alone or in combination, include visceral fat content, subcutaneous fat content, body mass index, weight, or blood pressure. As noted, the subject may be overweight or obese, or may have metabolic syndrome or an obesity-related condition. A determination as to these conditions may have been made by a physician or other health care professional (i.e., a subject may have been diagnosed as having one of these conditions or as being at risk therefor). As the present methods can be applied to maintain a condition (e.g., metabolic state, weight, or fat distribution), the subject may also be apparently healthy (e.g., with no sign of a metabolic disorder or weight disorder).

Where the subject has, or is at risk of developing, an obesity-related medical condition, the condition can be type 2 diabetes, cardiovascular disease (as evidenced, for example, by atherosclerosis), hypertension, arthritis (e.g., osteoarthritis or rheumatoid arthritis), cancer (e.g., breast cancer, a cancer of the esophagus or gastrointestinal tract (e.g., stomach cancer or colorectal cancer), endometrial cancer, or renal cell cancer), carpal tunnel syndrome, chronic venous insufficiency, daytime sleepiness, deep vein thrombosis, end stage renal disease, gallbladder disease, gout, liver disease, pancreatitis, sleep apnea, or urinary stress incontinence. The subject may also be a person who has had, or who is at risk of having, a cerebrovascular accident. Because these conditions are recognized as obesity-related medical conditions, a person who is overweight, and particularly grossly overweight or obese is, by virtue of that fact alone, at risk of developing one or more of these conditions.

Subjects amenable to treatment with the present methods may also have restricted mobility associated with, for example, joint pain, back pain, or paralysis. These circumstances may arise independently or may result from one or more obesity-related medical conditions. For example, joint pain or back pain may result from or may be exacerbated by arthritis.

The present methods can include assessing the levels of one or more of the parameters set out herein and comparing them on one or more occasions to recommended levels. An undesirable level can indicate that the subject would be amenable to treatment as described herein. In addition to the parameters described above, one can assess (e.g., to determine metabolic state) the subject's glucose tolerance, insulin resistance, visceral and/or subcutaneous fat content, weight, body mass index, and/or blood pressure. Such parameters can be assessed in the course of identifying a subject amenable to treatment and can be monitored at one or more times after treatment has begun. More specifically, a subject can be diagnosed as being overweight, being obese, having diabetes, being susceptible to adiposity, or having metabolic syndrome or a metabolic disease. The cause(s) of excess weight, when present, may be known or unknown. For example, patients suffering from weight gain and/or diabetes caused by restricted mobility (e.g., as a result of paralysis, arthritis, or a muscular or neurodegenerative disorder) or a drug (e.g., steroids, protease inhibitors, and/or antipsychotics used as a treatment of other maladies) can be treated with the methods described herein. As the invention is non-pharmacologically based, it is anticipated that it can also readily and safely be used to chronically suppress or delay the onset of childhood obesity, diabetes, or any other obesity-related medical condition. As noted, treating apparently healthy and/or non-overweight patients is within the scope of the present invention, and such treatment is applied to reduce the risk of weight gain, obesity, or a weight- or obesity-related condition.

Accordingly, the invention features methods of treating patients who are apparently healthy (e.g., patients who are not overweight, obese, diabetic or suffering from a metabolic syndrome or an obesity-related medical condition) to reduce the risk that they will develop a condition described herein, to delay its onset, or to impede its progression. Thus, “altering” a subject's metabolic state can be achieved by maintaining the subject's metabolic state or changing the expected progression as well as by improving one or more of the physiological parameters described herein. For example, patients who begin taking a steroid for treatment of other conditions often experience weight gain. The present methods can be applied to alter such a subject's metabolic state so that a given patient is less likely to gain weight or to gain less weight than expected. “Treating” a patient with the present methods encompasses improving their prognosis or expected outcome.

Considering the role of exercise in suppressing obesity and diabetes, it is widely accepted that exercise is effective because it metabolizes calories that accumulate through the diet and regulates insulin production through physiologic control of sugar in the bloodstream. Thus, one could conclude that the regulatory influence of exercise on suppressing the onset of obesity and diabetes is achieved through increasing calorie expenditure and reducing hyperglycemia, respectively, and thus the more strenuous the exercise, the greater the physiologic benefit. Our work, however, leads us to conclude that short daily bouts of extremely low-level mechanical, high-frequency loading can suppress fat production and improve insulin tolerance by controlling cellular differentiation. Because results can be achieved in a short time, the accumulation of a physical signal does not appear to be required, and this is consistent with the triggering of a biologic response. This trigger may change under systemic distress, such as endocrinopathy, obesity, cancers, infectious and/or genetic diseases, and/or aging, but by ensuring the trigger threshold is passed by adjusting duration, it still will not require an accumulated signal to obtain the benefit of the invention.

Because such low level signals, well below the forces, impacts, and/or accelerations that are generated by activities such as walking, are effective, the equilibration of caloric intake by metabolic work does not appear to be required. This is counterintuitive, counter to conventional wisdom, and implies a unique (or, at least, previously unappreciated) biologic mechanism. When we considered our results in view of how other physiologic systems, such as sight, hearing and touch, perceive exogenous signals through a frequency-selective “window,” and readily saturate when the signals are too high (too bright, too loud or too heavy), it occurred to us that physical signals could influence systems in a manner that is not necessarily dependent on reacting to highly intense—and perhaps dangerous—physical signals, but instead that cell processes are particularly sensitive to exogenous signals within specific frequency bands, and that exposure to such signals can control cellular outcomes, including differentiation of adipocyte precursors such as mesenchymal stem cells. We believe the physical signals we have used suppress adiposity not by stimulating the adipose tissue per se, but by influencing adipocyte precursors to differentiate into cells other than fat cells. Our studies indicate that the conditions described herein, including excess body weight, including weight gain to the point of obesity, metabolic state, and obesity-related medical conditions can be treated by the biologic suppression of adipocytic differentiation pathways and that that suppression can be achieved through low-level physical signals.

In addition to the methods carried out on whole, intact, living subjects, the signals described herein can be used to influence the fate of a cell in cell culture. These methods can be carried out by administering to the cell a low magnitude, high frequency mechanical signal on a periodic basis and for a time sufficient to influence the fate of the cell such that it differentiates into a cell type different from the cell type it would be expected to differentiate into in the absence of the signal (e.g., in the absence of a low magnitude, high frequency mechanical signal). Differentiation into a fully mature cell type may occur, but is not a necessary outcome.

Any cell type, including human cells of various types, can be subjected to the present signals. The methods can be applied, for example, to stem cells or progenitor cells (e.g., embryonic stem or progenitor cells or adult stem or progenitor cells, including mesenchymal stem cells).

According to another aspect of the present disclosure, the information that follows is based, in part, on our discovery that applying reasonably brief periods of low-magnitude, high-frequency mechanical signals (LMMS) to a cell (or population of cells, whether homogeneous or heterogeneous and whether found in cell culture, tissue culture, or within a living organism (e.g., a human)) on a periodic basis (e.g., a daily basis) can increase cellular proliferation and/or influence cell fate (i.e., influence one or more of the characteristics of a cell or alter the type of cell a precursor cell would have otherwise become).

The methods can be used to produce populations of cells, or to more quickly produce populations of cells, that can be used in various manufacturing processes. For example, the cells subjected to LMMS can be yeast cells used in any otherwise conventional process in the brewing industry. In other instances, the cells can be prokaryotic or eukaryotic cells used to produce therapeutic proteins (e.g., antibodies, other target-specific molecules such as aptamers, blood proteins, hormones, or enzymes). In other instances, the cells can be generated in cell or tissue culture for use in tissue engineering (e.g., by way of inclusion in a device, such as a scaffold, mesh, or gel (e.g., a hydrogel)).

Where the stimulus is applied in vivo, it may be applied to an organism from which tissue will be harvested (for, for example, use in a tissue engineering construct or for transplantation to a recipient). Alternatively, or in addition, the stimulus can be applied to a patient as a therapeutic treatment. The patient may have, for example, a damaged or defective organ or tissue. The damage or defect can be one that results from any type of trauma or it may be associated with nutritional deficiencies (e.g., a high fat diet). More generally, the patient can be any subject who would benefit from an increase in the number of stem cells within their tissues (e.g., an adult or elderly patient) or from an increase in the number of stem cells that differentiate into non-adipose cells. The signal can be applied to the patient by virtue of a platform on which the patient stands or lies. Alternatively, the signal can be applied more locally to a region or tissue of interest (e.g., by a handheld device).

The damaged or defective organs or tissues can include those affected by a wide range of medical conditions including, for example, traumatic injury (including injury induced in the course of a surgical or other medical procedure, such as an oncologic resection or chemotherapy), tissue damaging diseases, neurodegenerative diseases (e.g., Parkinson's Disease or Huntington's Disease), demyelinating diseases, congenital malformations (e.g., hypospadias), limb malformations, neural tube defects, and tissue loss, malfunction, or malformation resulting from or associated with an infection, compromised diet, or environmental insult (e.g., pollution or exposure to a damaging substance such as radiation, tar, nicotine, or alcohol). For example, the patient can have cardiac valve damage, tissue wasting, tissue inflammation, tissue scarring, ulcers, or undesirably high levels of adipose tissue (e.g., within the liver).

Accordingly, the invention features methods of increasing the proliferation and/or differentiation of a cell within the body of an organism (i.e., in vivo), a cell that has been removed from an organism and placed in culture, or a single-celled organism (e.g., a fungal or bacterial cell). A variety of cell types of diverse histological origins are amenable to the present methods. The cell can be a cell that has been removed from an organism and placed in culture for either a brief period (e.g., as a tissue explant) or for an extended length of time (e.g., an established cell line). The cell can be any type of stem cell, for example an embryonic stem cell or an adult stem cell. Adult stem cells can be harvested from many types of adult tissues, including bone marrow, blood, skin, gastrointestinal tract, dental pulp, the retina of the eye, skeletal muscle, liver, pancreas, and brain. The methods are not limited to undifferentiated stem cells and can include those cells that have committed to a partially differentiated state. More specifically, the cell can be a mesenchymal stem cell, a hematopoietic stem cell, an endothelial stem cell, or a neuronal stem cell. Such a partially differentiated cell may be a precursor to an adipocyte, an osteocyte, a hepatocyte, a chondrocyte, a neuron, a glial cell, a myocyte, a blood cell, an endothelial cell, an epithelial cell, a fibroblast, or a endocrine cell. Established cell lines, for example, embryonic stem cell lines, are also embraced by the methods, as are bacterial cells, including E. coli and other bacteria that can be used to produce recombinant proteins, and yeast (e.g., yeast suitable for brewing beer or other alcoholic beverages). Optionally, the cell can be one that naturally expresses a desirable gene product or that has been modified to express one or more exogenous genes. The methods can be applied to cells of mammalian origin (e.g., humans, mice, rats, canines, cows, horses, felines, and ovines) as well as cells from non-mammalian sources (e.g., fish and birds).

Regardless of the cell type that is used, the methods can be carried out by providing to the cell, or a subject in which the cell is found, a low-magnitude, high-frequency physical signal. For example, the signal can be supplied to increase or enhance the proliferation rate of a cell in culture. For example, a cell or a population of cells, whether homogenous or heterogeneous, may divide or double faster (e.g., 1-500% faster) than a comparable cell or population of cells, under the same or essentially similar circumstances, that has not been exposed to the present mechanical signals.

The signal can also be supplied to a whole organism to increase the proliferation rate of particular target cell populations. Because our data indicate these physical signals can influence the fate of mesenchymal stem cells, the present methods can also be used to help retain or restore any tissue type, with the likely exception of adipose tissue. For example, the present methods can be used to promote bone marrow viability and to direct the proliferation and controlled differentiation of stem cells, including those placed in cell culture, down specific pathways (e.g., toward differentiated bone cells, liver cells, or muscle cells, rather than toward adipocytes).

Any of the present methods can include the step of identifying a suitable source of cells and/or a suitable subject to whom the signal would be administered. Similarly, any of the present methods can be carried out using a human cell.

With respect to particular methods of treatment, the invention encompasses methods of treating a patient by administering to the patient a cell that has been treated, in culture or in a donor prior to harvesting, according to the methods described herein. More specifically, the methods encompass treating a patient who has experienced a traumatic injury to a tissue or who has a tissue damaging disease other than osteopenia or sarcopenia. The method can be carried out by administering to the patient a low magnitude, high frequency mechanical signal on a periodic basis and for a time sufficient to treat the injury or tissue damage. The patient can be, but is not necessarily, a human patient, and the traumatic injury can include a wound to the skin of the patient, such as a cut, burn, puncture, or abrasion of the skin. The traumatic injury can also include a wound to muscle, bone, or an internal organ. Where the injury is caused by disease, the disease can be a neurodegenerative disease.

Other patients amenable to treatment include those undergoing chemotherapy or radiation therapy, or those who have received a bone marrow transplant. Where tissue is transplanted, both the recipient patient and the tissue donor can be treated. The cells may also be treated in culture after harvest but prior to implantation. These methods can be carried out by administering to the patient a low magnitude, high frequency mechanical signal on a periodic basis and for a time sufficient to counteract a harmful side effect of the chemotherapy or radiation therapy on the patient's body or to improve the outcome of the bone marrow transplant. The side effect can be dry or discolored skin, palmar-plantar syndrome, damage to the skin caused by radiation or extravasation of the chemotherapeutic, hair loss, intestinal irritation, mouth sores or ulcers, cell loss from the bone marrow or blood, liver damage, kidney damage, lung damage, or a neuropathy.

The present methods can also be used to slow or reduce a sign or symptom of aging by administering to the patient a low magnitude, high frequency mechanical signal on a periodic basis and for a time sufficient to reduce the depletion of stem cells in the patient (as normally occurs with age). As with other methods described herein, the methods can be carried out on human patients, and elderly patients may be particularly amenable where the natural loss of stem cells occurs.

In another aspect, the invention features methods of preparing a tissue donor. The methods include administering to the donor a low magnitude, high frequency mechanical signal on a periodic basis and for a time sufficient to increase the number of cells in the tissue to be harvested for transplantation. The cells can be stem cells, and the tissue to be harvested can be bone marrow.

The effect of the physical signal on the rate of proliferation for a population of cells in culture can be assessed according to any standard manual or automated method in the art, for example, removing an aliquot of cells from the culture before and after treatment, staining the cells with a vital dye, e.g., trypan blue, and counting the cells in a hemacytometer, tetrazolium salt reagents such as MTT, XTT, MTS, fluorescence activated cell sorting, or Coulter counting. When the treatment is to a whole organism, an aliquot of cells can be removed using biopsy methods.

Where proliferation is enhanced in cell culture, the cells may be associated with a prosthetic or biomaterial. For example, the cells may be associated with a scaffold or substrate suitable for use as a graft, stent, valve, prosthesis, allograft, autograft, or xenograft.

The physical signal utilized with the methods of the present disclosure is preferably mechanical, but can also be another non-invasive modality (e.g., a signal generated by acceleration, electric fields, or transcutaneous ultrasound). The signal can be supplied on a periodic basis and for a time sufficient to achieve a desirable outcome (e.g., one or more of the outcomes described herein).

The time of exposure to the physical signal can be brief, and the periodic basis on which it is applied may or may not be regular. For example, the signal can be applied almost exactly every so many hours (e.g., once every 4, 8, 12, or 24 hours) or almost exactly every so many days (e.g., at nearly the same time every other day, once a week, or once every 10 or 14 days). Our expectation is that a positive outcome (e.g., an improved body weight, fat distribution, metabolic indicator, or obesity-related disease risk) will correlate with the level of compliance. However, less than ideal compliance and/or irregular application of the signal (which can be self-applied) are expected to be at least somewhat effective as well. Thus, in various embodiments, signals can be applied to a subject or cell daily, but at varied times of the day. Similarly, a subject or cell may miss one or more regularly scheduled applications and resume again at a later point in time. The length of time the signal (e.g., a mechanical signal) is provided can also be highly consistent in each application (e.g., it can be consistently applied for about 2-60 minutes, inclusive (e.g., for about 1, 2, 5, 10, 12, 15, 20, 25 or 30 minutes) or it can vary from one session to the next. Any of the methods can further include a step of identifying a subject (e.g., a human) prior to providing the low-magnitude, high-frequency physical (e.g., mechanical) signal, and the identification process can include an assessment of weight, fat mass, fat distribution, body mass index, blood sugar, triglyceride or free fatty acid levels, and/or any of other indicators of a metabolic state, as well as physical health and the disorder or tissue in need of repair. We may use the terms “subject,” “individual” and “patient” interchangeably. While the present methods are certainly intended for application to human patients, the invention is not so limited. For example, domesticated animals, including cats and dogs, or farm animals can also be treated.

The physical signals can be characterized in terms of magnitude and/or frequency, and are preferably mechanical in nature, induced through the weightbearing skeleton or directly by acceleration in the absence of weightbearing. Useful mechanical signals can be delivered through accelerations of about 0.01-10.0 g, where “g” or “1 g” represents acceleration resulting from the Earth's gravitational field (1.0 g=9.8 m/s/s). Surprisingly, signals of extremely low magnitude, far below those that are most closely associated with strenuous exercise, are effective. These signals can be, for example, of a lesser magnitude than those experienced during walking. Accordingly, the methods described here can be carried out by applying 0.1-1.0 g (e.g., 0.2-0.5 g (e.g., about 0.2 g, 0.3 g, 0.4 g, 0.5 g or signals therebetween (e.g., 0.25 g))). The frequency of the mechanical signal can be about 5-1,000 Hz (e.g., 20-200 Hz (e.g., 30-100 Hz)). For example, the frequency of the mechanical signal can be about 5-100 Hz, inclusive (e.g., about 50-90 Hz (e.g., 50, 60, 70, 80, or 90 Hz) or 20-50 Hz (e.g., about 20, 30, or 40 Hz). A combination of frequencies (e.g., a “chirp” signal from 20-50 Hz), as well as a pulse-burst of physical information (e.g., a 0.5 s burst of 40 Hz, 0.3 g vibration given at least or about every 1 second) can also be used. The duration of the signal application (i.e., the overall period of time the signal is applied) can be the same as that for intact subjects, but it may also vary from that (e.g., it may be shorter; the periodic basis can involve repetition of the signal every five to ten minutes, once or twice an hour, or on a daily or weekly basis). The magnitudes and frequencies of the acceleration signals that are delivered can be constant throughout the application (e.g., constant during a 10-minute application to a subject) or they may vary, independently, within the parameters set out herein. For example, the methods can be carried out by administering a signal of about 0.2 g and 20 Hz at a first time and a signal of about 0.3 g and 30 Hz at a second time. Further, distinct signals can be used for distinct purposes or aims, such as reversing an undesirable condition or preventing or inhibiting its development. For example, one can treat a subject for 15 minutes per day with a 0.3 g, 45 Hz signal where the aim is to lose fat mass, and for 10 minutes per day with a 0.2 g, 45 Hz signal to prevent fat gain.

While there are advantages to limiting the present methods to those that require purely physical stimuli, any of the present methods can be carried out in conjunction with other therapies, including those in which drug therapies are used to promote stem cell proliferation.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a graph showing the results of glucose tolerance tests in C3H.B6-6T obesity-prone mice and (control and treated with mechanical signal; mean±SD). The treated group was subjected to a signal of 0.2 g and 90 Hz for 15 minutes/day, 5 days per week. Glucose tolerance was analyzed at eight weeks into the protocol. There is a marked improvement in glucose tolerance after treatment.

FIG. 2 is a pair of images of a three-dimensional reconstruction of a region of the thoracic region of C3H.B6-6T obesity-prone mice (control and treated with mechanical signals). The treated group was exposed to mechanical signals at 0.2 g, 90 Hz for 15 minutes/day, 5 days per week, for 9 weeks. Fat content was determined two days before euthanasia. The amount of fat within the thoracic region is significantly lower in the treated mice.

FIG. 3 is a graph showing the results of a body mass analysis of BL6 control and mechanically treated mice fed a high-fat diet for 10 weeks. Ten-week-old male BL6 mice were treated for brief periods each day. There is a marked suppression of weight gain, despite the same food intake.

FIG. 4 is a pair of images of a coronal cross-sectional 3-D in vivo microCT scan of the abdominal region of a mechanically treated (VIB) and a control (CTRL) mouse after 11 weeks of whole body treatment (signal application) vs. control. As measured by microCT, VIB animals had 27.6% less body fat (subcutaneous and visceral) in the torso than CTRL (p<0.005). VIB had 22.5% less epididymal and 19.5% less subcutaneous fat than CTRL (p<0.01).

FIG. 5 is a graph depicting body mass (g) of control and vibrated mice (n=20 in each group) over the span of twelve weeks. No significant differences in average body mass were measured between the controls and vibrated animals. The vibrated animals were vibrated five days per week, fifteen minutes per day at a 90 Hz, 0.4 g peak-to-peak acceleration.

FIG. 6A is an image of a three-dimensional longitudinal reconstruction of subcutaneous and epididymal fat content through the midsection of the torso of a control mouse, performed in vivo at twelve weeks, using computed tomographic signal parameters specifically sensitive to fat.

FIG. 6B is an image of a three-dimensional longitudinal reconstruction of subcutaneous and epididymal fat content through the midsection of the torso of a vibrated mouse (vibrated five days per week, fifteen minutes per day at a 90 Hz, 0.4 g peak-to-peak acceleration), performed in vivo at twelve weeks, using computed tomographic signal parameters specifically sensitive to fat.

FIG. 6C is an image of a three-dimensional transverse reconstruction of subcutaneous and epididymal fat content through the midsection of the torso of a control mouse, performed in vivo at twelve weeks, using computed tomographic signal parameters specifically sensitive to fat.

FIG. 6D is an image of a three-dimensional transverse reconstruction of subcutaneous and epididymal fat content through the midsection of the torso of a vibrated mouse (vibrated five days per week, fifteen minutes per day at a 90 Hz, 0.4 g peak-to-peak acceleration), performed in vivo at twelve weeks, using computed tomographic signal parameters specifically sensitive to fat. The data presented in FIGS. 6A-6D shows that following twelve weeks of daily, 15 minute low-level mechanical signal, the average amount of fat within the torso is 26% lower than that of age-matched control animals.

FIG. 7A is a graph depicting fat volume as a function of body mass for the control mice (n=15). The control animals demonstrated a strong positive correlation between fat volume and weight (r²=0.70; p=0.0001).

FIG. 7B is a graph depicting fat volume as a function of body mass for vibrated mice (n=15; vibrated five days per week, fifteen minutes per day at a 90 Hz, 0.4 g peak-to-peak acceleration). The vibrated animals showed a weak correlation between fat volume and weight (r²=0.18; p=0.1). Considering identical food intake between groups represented in FIGS. 7A and 7B, the data in FIGS. 7A and 7B indicate that the mechanical signals suppressed adipogenesis.

FIG. 8A is a graph depicting the level of total triglycerides (mg) in adipose tissue in vibrated mice (dark grey) and control group (light grey). The vibrated animals were vibrated five days per week, fifteen minutes per day at a 90 Hz, 0.4 g peak-to-peak acceleration. Triglycerides were 21.2% lower in adipose tissue in the vibrated animals when compared with controls (p=0.3; n=8 in each group). Mean and standard deviations are shown.

FIG. 8B is a graph depicting the level of total triglycerides (mg) in liver in vibrated mice (dark grey) and control group (light grey). The vibrated animals were vibrated five days per week, fifteen minutes per day at a 90 Hz, 0.4 g peak-to-peak acceleration. Triglycerides were 39.1% lower in livers of the vibrated animals when compared with controls (p=0.022; n=12 in each group). Mean and standard deviations are shown.

FIG. 8C is a graph depicting the level of total non-esterified fatty acids (mmol) in adipose tissue in vibrated mice (dark grey) and control mice (light grey). The vibrated animals were vibrated five days per week, fifteen minutes per day at a 90 Hz, 0.4 g peak-to-peak acceleration. Non-esterified fatty acids were 37.2% lower in adipose tissue of the vibrated animals when compared with controls (p=0.014; n=8 in each group). Mean and standard deviations are shown.

FIG. 8D is a graph depicting the level of total non-esterified fatty acids (mmol) in livers of vibrated mice (dark grey) and control mice (light grey). The vibrated animals were vibrated five days per week, fifteen minutes per day at a 90 Hz, 0.4 g peak-to-peak acceleration. Non-esterified fatty acids were 42.6% lower in livers of the vibrated animals when compared with controls (p=0.023; n=12 in each group). Mean and standard deviations are shown.

FIG. 9 is images of the pancreatic tissue of mice fed a high fat diet for 13 weeks. The top left image is a histology slide of the pancreatic tissue of a control mouse (Non-LIV, Non-Inj), and the top right image is a histology slide of the pancreatic tissue of a mouse that was mechanically treated with low intensity vibration (LIV, Non-Inj). The bottom left image is a histology slide of the pancreatic tissue of a control mouse that was injected with cytokines to induce pancreatitis (Non-LIV, IL12+IL18 Inj), and the bottom right image is a histology slide of a mechanically treated mouse that was injected with cytokines (LIV, IL12+IL18 Inj). No significant differences in appearance of the Non-LIV, Non-Inj and LIV, Non-Inj mice were observed, while Non-LIV, IL12+IL18 Inj mice showed severe inflammation and tissue damage compared to the LIV, IL12+IL18 Inj mice.

FIG. 10A is a dot plot from a flow cytometry analysis of stem cells in general (Sca-1 single positive, upper quadrants), and MSCs specifically (both Sca-1 and Pref-1 positive, upper right quadrant) in the bone marrow of a control mouse.

FIG. 10B is a dot plot from a flow cytometry analysis of stem cells in general (Sca-1 single positive, upper quadrants), and MSCs specifically (both Sca-1 and Pref-1 positive, upper right quadrant) in the bone marrow of a vibrated mouse.

FIG. 10C is a graph comparing the total stem cell number, calculated as % positive cells/total cells for the cell fraction showing highest intensity staining, in a control (CON) to and vibrated (LMMS) mouse.

FIG. 10D is graph comparing the mesenchymal stem cell number, calculated as % positive cells/total cells for the cell fraction showing highest intensity staining, in a control (CON) and vibrated (LMMS) mouse.

FIG. 11A shows distinct cell populations identified in flow cytometry, with stem cells being identified as low forward (FSC) and side (SSC) scatter.

FIG. 11B is a graph showing osteoprogenitor cells, identified as Sca-1(+) cells, residing in the region highlighted as high FSC and SSC, and were 29.9% (p=0.23) more abundant in the bone marrow of LMMS treated animals.

FIG. 11C is a graph showing that the preadipocyte population, identified as Pref-1 (+), Sca-1 (−), demonstrated a trend (+18.5%; p=0.25) towards an increase in LMMS relative to CON animals (CON).

FIG. 12A is a graph showing real time RT-PCR analysis of bone marrow samples harvested from untreated (CON) mice and mice subject to 6 weeks LMMS treatment. The osteogenic gene Runx2 was significantly upregulated in the LMMS-treated mice.

FIG. 12B is a graph showing real time RT-PCR analysis of bone marrow samples harvested from untreated (CON) mice and mice subject to 6 weeks LMMS treatment. The adipogenic gene PPARγ was downregulated in the LMMS-treated mice.

FIG. 13A is a graph showing bone volume fraction, as measured in vivo by low resolution μCT, in control (CON) and vibrated (LMMS) mice. LMMS increased bone volume fraction across the entire torso of the animal.

FIG. 13B is a graph showing post-sacrifice, high resolution CT of the proximal tibia in control (CON) and vibrated (LMMS) mice. LMMS significantly increased trabecular bone density.

FIG. 13C is a representative μCT reconstruction at the proximal tibia in a control (CON) mouse.

FIG. 13D is a representative μCT reconstruction at the proximal tibia in a vibrated (LMMS) mouse. Tibiae from LMMS mice showed enhanced morphological properties.

FIG. 14A shows in vivo μCT images used to discriminate visceral and subcutaneous adiposity in the abdominal region of a CON and LMMS mouse. Visceral fat is shown in dark grey, subcutaneous fat in light gray.

FIGS. 14B, 14C, 14D and 14E show linear regressions of calculated visceral adipose tissue (VAT) volume against adipose TG, adipose NEFA, liver TG and liver NEFA, respectively. Linear regressions of calculated visceral adipose tissue (VAT) volume against adipose and liver biochemistry values demonstrated strong positive correlations in CON, and weak correlations in LMMS, as well as generally lower levels for all LMMS biochemical values. N=6 for adipose (FIGS. 14B and 14C), N=10 for liver (FIGS. 14D and 14E). Regressions for adipose TG (p=0.002), adipose NEFA (p=0.03), liver TG (p=0.006) and liver NEFA (p=0.003) were significant for CON animals, but only liver NEFA (p=0.02) was significant for LMMS. Overall, LMMS mice exhibited lower, non-significant correlations in liver TG (p=0.06), adipose TG (p=0.19), and adipose NEFA (p=0.37) to increases in visceral adiposity.

FIG. 15A shows reconstructed in vivo μCT images of total body fat (dark grey) in untreated (CON) and vibrated (LMMS) mice.

FIG. 15B is a graph showing the effect of LMMS treatment on fat volume in two mouse models of obesity. In one, “fat diet”, mice were placed on a high fat diet at the same time that LMMS treatment was initiated. After 12 weeks, mice that received LMMS exhibited 22.2% less fat volume as compared to control mice (CON) that did not receive LMMS treatment. In the other model, “obesity”, mice were maintained on a high fat diet for 3 weeks prior to LMMS treatment. No reduction of fat volume was observed in LMMS mice in the “obesity” model.

FIG. 15C is a graph showing the effect of LMMS treatment on percent adiposity the mouse models shown in FIG. 15B. In the “fat diet” model the percent adiposity, calculated as the relative percentage of fat to total animal volume, LMMS reduced the percent animal adiposity by 13.5% (p=0.017); no effect was observed in the “obesity” model. The lack of a response in the already obese animals suggests that the mechanical signal works primarily at the stem cell development level, as existing fat is not metabolized by LMMS stimulation. Suppression of the obese phenotype was achieved to a degree by stem cells preferentially diverting from an adipogenic lineage.

FIG. 16 is a graph depicting changes in bone density, muscle area and fat area in a group of young osteopenic women subject to LMMS for one year. As measured by CT scans in the lumbar region of the spine, a group of young osteopenic women subject to LMMS for one year (n=24; gray bars±s.e.) increased both bone density (p=0.03 relative to baseline; mg/cm3) and muscle area (p<0.001; cm2), changes which were paralleled by a non-significant increase in visceral fat formation (p=0.22; cm2). Conversely, women in the control group (n=24; white bars±s.e.), while failing to increase either bone density (p=0.93) or muscle area (p=0.52), realized a significant increase in visceral fat formation (p=0.015).

FIG. 17A is a reconstruction of in vivo CT data through longitudinal section of mice showing difference in fat quantity and distribution in CON and LMMS mice. Image represents total body fat in dark gray.

FIG. 17B is a graph showing fat volume in control (CON) and vibrated (LMMS) mice. Total fat volume was decreased by 28.5% (p=0.030) after 12 weeks of daily treatment with LMMS.

FIG. 17C graph showing epididymal fat pad weight at sacrifice in the control (CON) and vibrated (LMMS) mice of FIG. 17A.

FIG. 18A is an image of high resolution scans of the proximal tibia (600 mm region, 300 mm below growth plate) done ex vivo demonstrate the anabolic effect of low magnitude, high frequency mechanical stimulation to bone.

FIG. 18B is a graph showing bone volume fraction in control (CON) and LMMS treated mice. LMMS animals showed significant enhancements in bone volume fraction.

FIG. 18C is a graph showing trabecular number in control (CON) and LMMS treated mice. LMMS animals showed significant enhancements in trabecular number.

FIG. 19A and FIG. 19B are representative dot plots from flow cytometry experiments demonstrating the ability of LMMS to increase the number of cells expressing Stem Cell Antigen-1 (Sca-1). Cells in this experiment were double-labeled with Sca-1 (to identify MSCs, y-axis) and Preadipocyte factor (Pref-1, x-axis) to identify preadipocytes. Sca-1 only cells (highlighted, upper left) represent the population of uncommitted stem cells.

FIG. 19C is a graphical representation of the data in FIG. 19A and FIG. 19B. The actual increase in stem cell number was calculated as % positive cells/total number of bone marrow cells. RD denotes an age-matched control group of animals fed a regular diet, FD denotes fat diet fed animals. Regardless of diet, LMMS treatment increases the number of Sca-1 positively labeled cells.

FIG. 20A is a graph showing the percentage of GFP positive cells harvested from various tissues in control (CON) or vibrated (LMMS) mice. LMMS treatment was administered for 6 weeks. (N=8). (FIG. 20B) The reduced ratio of adipocytes shown relative to bone marrow GFP expression in LMMS indicates reduced commitment to fat. Ratio of adipocytes to blood is shown as a constant engraftment control.

FIG. 21A-21D are images and graphs of lacerated gastrocnemius muscle of non-vibrated and vibrated mice. The tissue shown in FIG. 21A have been stained for visualization of the muscle fibers of the lacerated tissue in each of the non-vibrated and vibrated mice, with FIG. 21B graphically illustrating the fiber area in the non-vibrated and vibrated mice of FIG. 21A. The tissue shown in FIG. 21C have been stained for visualization of collagen deposition in the lacerated tissue in each of the non-vibrated and vibrated mice, with FIG. 21D graphically illustrating the percentage of area collagen in the non-vibrated and vibrated mice of FIG. 21C. The fiber area in the lacerated muscle of the vibrated mice is greater than the fiber area of the non-vibrated mice, while the percentage of area collagen in the lacerated muscle of the vibrated mice is less than that of the non-vibrated mice.

FIG. 22A is images of the wounded tissue of non-vibrated and vibrated mice. The top row of images show re-epithelialization and granulation tissue thickness in the non-vibrated and vibrated mice, and the bottom row of images show angiogenesis associated with cell marker CD31 in the non-vibrated and vibrated mice.

FIG. 22B are graphs illustrating the percentage of endothelial cell marker CD31 staining, the granulation tissue thickness, and the percentage of re-epithelialization, respectively, in the non-vibrated (control) and vibrated mice of FIG. 22A.

FIG. 23 is a schematic illustration of a platform for applying physical signals to a subject in accordance with an embodiment of the present disclosure.

### DETAILED DESCRIPTION

Described below are methods of the present disclosure for applying physical stimuli to subjects. These methods can be applied in, and are expected to benefit subjects in, a great variety of circumstances that arise in the context of, for example, maintaining or improving the subject's metabolic state. The methods can be carried out, for example, to affect overt manifestations of the metabolic state (e.g., to suppress weight gain, obesity and defined conditions such as diabetes), and they may also affect underlying physiological events (e.g., the suppression of free fatty acids and triglycerides in adipose, muscle and liver tissue or the maintenance of “healthy” levels of such agents).

These methods can also be applied in, and are expected to benefit subjects in, a great variety of circumstances that arise in the context of, for example, traumatic injury (including that induced by surgical procedures), wound healing (of the skin and other tissues), cancer therapies (e.g., chemotherapy or radiation therapy), tissue transplantation (e.g., bone marrow transplantation), and aging. More generally, the present methods apply where patients would benefit from an increase in the number of cells (e.g., stem cells) within a given tissue and, ex vivo, where it is desirable to increase the proliferation of cells (e.g., stem cells) for scientific study, inclusion in devices bearing cells (e.g., polymer or hydrogel-based implants), and administration to patients.

### I. Methods of Maintaining or Improving the Metabolic State of a Subject

The methods of the present disclosure are based, inter alia, on our findings that even brief exposure to high frequency, low magnitude physical signals (e.g., mechanical signals), inducing loads below those that typically arise even during walking, have marked effects on suppressing adiposity, triglyceride and free fatty acid production, and provide a unique, non-pharmacologic intervention for the control of weight gain, obesity, diabetes, and other obesity-related medical conditions. The marked response to low and brief signals in the phenotype of a growing animal suggests the presence of an inherent physiologic process that has been previously unrecognized.

### Metabolic State

Metabolism constitutes a series of chemical processes that occur inside living organisms, including single cells found in vivo or placed in cell culture, which are necessary to maintain energy and life. In regard to the higher order organisms, such as a humans, the metabolic state of a subject can be affected by, for example, the subject's having metabolic syndrome or a metabolic disease, being overweight or obese, being inactive, confined to bed, or having diabetes or another obesity-related medical condition. Conversely, a poor metabolic state can lead to restricted mobility or even paralysis.

A subject's metabolic state can be reflected by the level of one or more of the following components in the subject (e.g., in a sample obtained from the subject (e.g., from the bloodstream, urine, protoplasm and/or tissue)): triglycerides, free fatty acids, cholesterol, fibrinogen, C-reactive protein, hemoglobin Alc, insulin, and various cytokines (e.g., adipokines such as leptin (Ob ligand), adiponectin, resistin, plasminogen activator inhibitor-1 (PAI-1), tumor necrosis factor-alpha (TNFα) and visfatin), including pro-inflammatory cytokines. Adipokines are believed to have a role in modifying appetite, insulin resistance and atherosclerosis, and they may be modifiable causes of morbidity in people with obesity. A subject's metabolic state can also be reflected by glucose tolerance, insulin resistance, fat content (e.g., visceral or total fat), weight, body mass index, and/or blood pressure.

The present methods require application of a signal to a subject, and they can also, optionally, include a step of identifying a suitable subject. This step is optional because our research indicates that virtually anyone can benefit from the present methods, which can help maintain (i.e., impede a worsening of) the subject's current metabolic state, and that is true of subjects who are in excellent health. Where a subject's metabolic state is “reflected by” a given physiological parameter (or parameters), that parameter (or those parameters) can be evaluated, quantitatively or qualitatively, and this assessment can be used as a further indication of which subjects may be most likely to immediately benefit from the present methods or benefit to a greater extent. For example, where a subject's quality of life is negatively impacted by excessive weight, and where the present methods reduce or help to reduce that weight, that subject would be more immediately benefited than (and more greatly benefited than), for example, a subject who is only slightly overweight or who has been able to maintain a healthy weight.

The methods described here can be used to maintain or improve the metabolic state and are carried out by providing, to the subject, a low-magnitude and high-frequency physical signal, such as a mechanical signal. As noted, the physical signal can be administered other than by a mechanical force (e.g., an ultrasound signal that generates the same displacement can be applied to the subject), and the signal, regardless of its source, can be supplied (or applied or administered) on a periodic basis and for a time sufficient to maintain, improve, or inhibit a worsening of the metabolic state generally or to maintain, improve, or inhibit a worsening of a specific condition described herein (e.g., insulin resistance, obesity, diabetes or other obesity-related medical condition, or adipogenesis).

Subjects with Metabolic Syndrome

Metabolic syndrome, which is also called obesity syndrome, syndrome X, or insulin resistance syndrome, presents as a combination of metabolic risk factors. These factors include: weight gain, hypertension, atherogenic dyslipedemia (blood fat disorders, such as high triglycerides, low and/or high density lipoproteins (LDL and/or HDL); high LDL cholesterol fosters plaque buildup in arteries), insulin resistance or glucose intolerance, pro-thrombotic state (e.g., high fibrinogen or plasminogen activator inhibitor-1 in the blood) and pro-inflammatory state (e.g., elevated C-reactive protein in the blood). Accordingly, any of these factors can be assessed as a relevant physiological parameter. Amounts of each of the substances listed above (e.g., LDLs) that are considered normal, or healthy, are known in the art. These amounts are usually specified within a range. Similarly, tests and methods for assessing the parameters listed above (e.g., glucose tolerance or intolerance and weight gain) are known in the art, and the results are recognizable by health care professionals as desirable (healthy) or undesirable (indicating a disease process (e.g., diabetes)) or unhealthy metabolic state, including obesity.

Potential causes of metabolic syndrome include physical inactivity, aging, hormonal imbalance and genetic predisposition. Thus, these causes may also be considered when performing the present methods and considering or evaluating subjects for treatment. Left uncontrolled, metabolic syndrome can lead to increased risk of diabetes and heart disease. Where a patient is also obese, that patient is at risk of developing an obesity-related medical condition. Recommended management of the syndrome presently focuses on lifestyle changes such as weight loss, increased physical activity and healthy eating habits. Any of these can be practiced in connection with the present methods, as can any treatment for an obesity-related medical condition.

The methods described here can be used to maintain, improve, or prevent (e.g., by inhibiting onset) a condition described herein (e.g., to maintain a healthy weight or to improve a sign or symptom of an undesirable state, such as metabolic syndrome or an obesity-related medical condition) by providing to a subject a low-magnitude and high-frequency physical (e.g., mechanical) signal on a periodic basis. The signal is applied for a time sufficient to maintain, improve, or prevent the condition (e.g., to maintain a healthy weight or to improve a sign or symptom of metabolic syndrome or an obesity-related medical condition). As noted, the physical signal is believed to reduce or suppress adipogenesis, and it may do so by influencing cellular differentiation toward a non-adipocyte fate). As also noted, the methods can include a step of assessing one or more of the physiological parameters described above in order to identify a subject amenable to treatment (e.g., hormonal imbalance). The subject can present with evidence of metabolic syndrome or as apparently healthy (e.g., a subject can have normal insulin sensitivity and blood glucose but a family history of diabetes or a genetic predisposition to obesity, as described further below). Moreover, the methods described herein can serve to suppress the negative sequelae associated with dyslipedemia and obesity, including atherosclerosis, congestive heart failure, myocardial infarction, hypertension, sleep apnea, and arthritis.

### Subjects Who are Overweight or Obese

Generally, an individual is considered to be overweight if his or her weight is 10% higher than normal as defined by a standard height/weight chart. An individual is considered to be obese if his or her weight is 30% or more above what is considered normal by the height/weight chart or as calculated relative to an ideal Body Mass Index (BMI).

Obesity is characterized by an excessively high amount of body fat or adipose tissue. This condition is common and varies from individual to individual. Some differences among individuals are influenced by inherited genetic variations. Genetic factors have been implicated in maintenance of body weight and effectiveness of diet and exercise, and some of the genes that have been implicated in predisposition to obesity include: UCP2 (whose gene product regulates body temperature), LEP (whose gene product, leptin, acts on the hypothalamus to reduce appetite and increase the body's metabolism), LEPR (leptin receptor), PCSK1 (whose gene product, proprotein convertase subtilisin/kexin type 1, processes hormone precursors such as POMC), POMC (whose gene product, among other functions, stimulates adrenal glands), MC4R (whose gene product is a melanocortin 4 receptor) and Insig2 (whose gene product regulates fatty acid and cholesterol synthesis). Other genes, which have been associated or linked with human obesity phenotypes now number above 200. Obesity gene map databases are available on the worldwide web and genes and gene maps are described in the scientific literature (see, e.g., Perusse et al., Obesity Res. 13:381-490, 2005). Any of these factors can be taken into consideration when determining a subject's risk of obesity.

Obesity affects an individual's quality of life and carries an increased risk for several related syndromes that can reduce life expectancy. Obese children are more prone to develop Type 2 diabetes (Cara et al., Curr. Diab. Rep. 6:241-250, 2006), while overweight adults, not yet even obese, are more susceptible to chronic, debilitating diseases and increased risk of death (Adams, NEJM, NEJMoa055643, 2006). Due to dyslipedemia and hypercholesterolemia, obese individuals have a markedly increased risk of atherosclerosis, leading to coronary artery disease and myocardial infarction. In addition, a vast majority of obese individuals have associated hypertension that can lead to thickening of the left ventricular wall (left ventricular hypertrophy), a leading cause of congestive heart failure. It is also well-established that obesity is associated with a generalized inflammatory response, which in combination with the increased mass of an individual puts mechanical and immunological stress on the major joints in the body, leading to more severe and earlier onset of arthritis. Further, nearly all obese individuals display various degrees of sleep apnea, a condition in which normal breathing is interrupted during periods of sleep, resulting in oxygen depletion, restless sleep, and chronic fatigue. While exercise remains the most readily available and generally accepted means of curbing weight gain and the onset of type II diabetes, compliance is poor. As described elsewhere herein, by reducing obesity or the risk of obesity, the present methods also reduce obesity-related medical conditions or the risk thereof.

Although obesity results in states of dyslipidemia, lipodystrophy (the absence of adipose tissue deposits) can have the same negative consequence due to limited peripheral nonesterified free fatty acids (NEFA) and triglyceride storage capacity (Petersen and Shulman, Am. J. Med. 119:S10-S16, 2006). Thus, a physiologic balance between lipid storage and lipid release must be maintained for optimum metabolism. The ability to suppress adipose tissue expansion by mechanical signals described herein, as well as to limit NEFA and triglyceride production (see, e.g., Example 3 infra), may provide a simple, non-pharmacologic approach to limit obesity in a manner sufficient to prevent the consequences of dyslipidemia.

The methods described herein can be used to treat an overweight or obese subject by providing to the subject a low-magnitude, high-frequency physical signal, preferably mechanical in origin, on a periodic basis and for a time sufficient to maintain or improve the subject's condition (e.g., reduce or suppress adipogenesis). In identifying a subject amenable to treatment, the methods can include a step of analyzing one or more of the genes listed or referenced above, or of assessing a subject's weight or predisposition for obesity by other methods known in the art. Because the signal does not required drug administration to be effective, this treatment described herein can also be safely administered to a juvenile and young-adult population to suppress childhood obesity and/or juvenile diabetes.

Subjects with Diabetes or Other Obesity-Related Medical Conditions

Diabetes mellitus is a disease in which the body does not produce or properly use insulin, a hormone that converts sugar, starches and other foods into energy. People with diabetes have a high circulating blood sugar level. Both genetics and environmental factors, such as obesity and lack of exercise, can play a role in the development and pathogenesis of diabetes.

There are generally considered to be four major types of diabetes: Type 1, Type 2, gestational and pre-diabetes. Type 1 Diabetes is an autoimmune disorder and results from the body's failure to produce insulin. Type 2 Diabetes results from the body's developed resistance to insulin, combined with relative insulin deficiency. Gestational diabetes affects pregnant women. Pre-diabetes is a condition in which a person's blood glucose levels are higher than normal but not high enough for a diagnosis of Type 2 Diabetes.

About 18 regions of the genome have been linked with Type 1 Diabetes risk (see, e.g., Dean et al., “The Genetic Landscape of Diabetes”, which is published online by the National Center for Biotechnology Information (NCBI)). These regions, each of which may contain several genes, have been labeled IDDM1 to IDDM18. The most well-studied is IDDM1, which contains the HLA genes that encode immune response proteins. There are two other non-HLA genes which have been identified thus far. One, IDDM2, is the insulin gene, and the other maps close to CTLA4, which has a regulatory role in the immune response.

Development of Type 2 Diabetes is associated with both genetics and environmental factors (see Dean et al.). Some genes implicated in developing Type 2 Diabetes encode: the sulfonylurea receptor (ABCC8), the calpain 10 enzyme (CAPN10), the glucagon receptor (GCGR), the enzyme glucokinase (GCK), the glucose transporter (GLUT2), the transcription factor HNF4A, the insulin hormone (INS), the insulin receptor (INSR), the potassium channel KCNJ11, the enzyme lipoprotein lipase (LPL), the transcription factor PPARgamma, the regulatory subunit of phosphorylating enzyme (PIK3R1) and others. These genes can be evaluated when identifying a subject who may benefit from the present methods.

Low-level mechanical signals described herein (see, e.g., Example 3 infra), can result in lower adiposity and suppress the production of nonesterified free fatty acids (NEFA) and triglycerides, key biochemical factors related to Type 2 diabetes. Numerous studies have demonstrated that dyslipidemia can have major negative impact on metabolism, growth and development. In particular, intra-tissue lipid accumulation (liver steatosis) and intra-myocellular lipids have been closely linked to insulin resistance and is the best predictor for the future development of insulin resistance (Unger, Endocrinology 144:5159-65, 2003).

The methods of the invention can be used to maintain or improve symptoms of diabetes in a subject by providing to the subject a low-magnitude, high-frequency physical signal, preferably a mechanical signal, at least once and preferably on a periodic basis and for a time sufficient to maintain or improve diabetes (e.g., by reducing or suppressing adipogenesis). In identifying a subject amenable to treatment, the methods can include a step of analyzing one or more of the genes listed or referenced above, of assessing a subject's blood glucose, or by other methods known in the art for identifying a patient who is diabetic or pre-diabetic. Similarly to the prevention and treatment of obesity, because this treatment is not based on the use of drugs, it can safely be used as an intervention in pre-adolescents and adolescents in the prevention and treatment of juvenile diabetes.

A subject who has been diagnosed as having, or is at risk of developing, another obesity-related medical condition can be treated as described herein. Other obesity-related medical conditions include cardiovascular disease, hypertension, osteoarthritis, rheumatoid arthritis, breast cancer, a cancer of the esophagus or gastrointestinal tract, endometrial cancer, renal cell cancer, carpal tunnel syndrome, chronic venous insufficiency, daytime sleepiness, deep vein thrombosis, end stage renal disease, gallbladder disease, gout, liver disease, pancreatitis, sleep apnea, a cerebrovascular accident, and urinary stress incontinence.

Pancreatitis, for example, is characterized by inflammation of the pancreas. The pathogenesis of pancreatitis involves multiple mechanisms that participate in the development of inflammation, necrosis, and/or fibrosis. Acute pancreatitis involves inflammation of the pancreas that is usually accompanied by abdominal pain, whereas in chronic pancreatitis inflammation may resolve, but the gland may be damaged by fibrosis, calcification, and ductal inflammation. Subjects with acute pancreatitis may have elevated levels of interleukin-12 (IL-12) and interleukin-18 (IL-18) cytokines, and IL-18 levels have been shown to be high in obese subjects. Insulin resistance has also been shown to co-exist with chronic pancreatitis. Damage to the pancreas may also by affected by a wide range of other medical conditions, e.g., traumatic injury or environmental insult, as discussed above.

The methods of this invention can be used to ameliorate the severity of pancreatitis in a subject by providing to the subject a low-magnitude, high frequency physical signal (e.g., a mechanical signal) on a period basis for a time sufficient to reduce or suppress pancreatitis. Subjects amendable to this treatment include those diagnosed with being insulin resistant, overweight or obese, and at risk of being overweight or obese. The subjects can also be those diagnosed as having diabetes or metabolic syndrome

### Adipogenesis

Adipogenesis, also called lipogenesis, is the formation of fat, including transformation of nonfat food materials into body fat. Adipogenesis also refers to the development of fat cells from preadipocytes.

The methods of this invention can be used to suppress or reduce adipogenesis in a subject (e.g., a human) by providing to the subject a low-magnitude, high-frequency physical signal (e.g., a mechanical signal) on a periodic basis and for a time sufficient to reduce or suppress adipogenesis. Subjects amenable to this treatment can include those diagnosed with being insulin resistant, overweight or obese, and at risk of being overweight or obese. The subjects can also be those diagnosed as having diabetes or metabolic syndrome.

### II. Methods of Increasing the Proliferation and/or Differentiation of Cells

The methods are based, inter alia, on our findings that even brief exposure to high frequency, low magnitude physical signals (e.g., mechanical signals), inducing loads below those that typically arise even during walking, have marked effects on the proliferation and differentiation of cells, including stem cells such as mesenchymal stem cells. The marked response to low and brief signals in the phenotype of a growing animal suggests the presence of an inherent physiologic process that has been previously unrecognized.

More specifically, we have found that non-invasive mechanical signals can markedly elevate the total number of stem cells in the marrow, and can bias their differentiation towards osteoblastogenesis and away from adipogenesis, resulting in both an increase in bone density and less visceral fat. A pilot trial on young osteopenic women suggests that the therapeutic potential of low magnitude mechanical signals can be translated to the clinic, with an enhancement of bone and muscle mass, and a concomitant suppression of visceral fat formation.

Described herein are methods and materials for the use of low magnitude mechanical signals (LMMS), of a specific frequency, amplitude and duration, that can be used to enhance the viability and/or number of stem cells (e.g., in cell culture or in vivo), as well as direct their path of differentiation. The methods can be used to accelerate and augment the process of tissue repair and regeneration, help alleviate the complications of treatments (e.g., radio- and chemotherapy) which compromise stem cell viability, enhance the incorporation of tissue grafts, including allografts, xenografts and autografts, and stem the deleterious effects of aging, in terms of retaining the population and activity of critical stem cell populations.

### Stem Cells

The methods of the invention can be used enhance or increase proliferation (as assessed by, e.g., the rate of cell division), of a cell and/or population of cells in culture. The cultured population may or may not be purified (i.e., mixed cell types may be present, as may cells at various stages of differentiation). Numerous cell types are encompassed by the methods of the invention, including adult stem cells (regardless of their tissue source), embryonic stem cells, stem cells obtained from, for example, the umbilical cord or umbilical cord blood, primary cell cultures and established cell lines. Useful cell types can include any form of stem cell. Generally, stem cells are undifferentiated cells that have the ability both to go through numerous cycles of cell-division while maintaining an undifferentiated state and, under appropriate stimuli, to give rise to more specialized cells. In addition, the present methods can be applied to stem cells that have at least partially differentiated (i.e., cells that express markers found in precursor and mature or terminally differentiated cells).

Adult stem cells have been identified in many types of adult tissues, including bone marrow, blood, skin, the gastrointestinal tract, dental pulp, the retina of the eye, skeletal muscle, liver, pancreas, and brain. Bone marrow is an especially rich source of stem cells and includes hematopoietic stem cells, which can give rise to blood cells, endothelial stem cells, which can form the vascular system (arteries and veins) and mesenchymal stem cells. Mesenchymal stem cells, also referred to as MSCs, marrow stromal cells, multipotent stromal cells, are multipotent stem cells that can differentiate into a variety of cell types, including osteoblasts, chondrocytes, myocytes, adipocytes, and beta-pancreatic islet cells.

The methods of the invention can also be used to enhance or increase the proliferation of cultured cell lines, including but, not limited to embryonic stem cell lines, for example, the human embryonic stem cell line NCCIT; the mouse embryonic stem cell line R1/E; mouse hematopoeitic stem cell line EML Cell Line, Clone 1. Such cell lines can be obtained from commercial sources or can be those generated by the skilled artisan from tissue samples or explants using methods known in the art. The origins of any given cell line can be analyzed using cell surface markers, for example, Sca-1 or Pref-1, or molecular analysis of gene expression profiles or functional assays.

The methods described here can be carried out by providing, to the subject, a low-magnitude and high-frequency physical signal, such as a mechanical signal. The physical signal can be administered other than by a mechanical force (e.g., an ultrasound signal that generates the same displacement can be applied to the subject), and the signal, regardless of its source, can be supplied (or applied or administered) on a periodic basis and for a time sufficient to maintain, improve, or inhibit a worsening of a population of cells (e.g., the proliferation of MSCs in culture).

### III. Low-Magnitude High-Frequency Mechanical Signals

The treatments disclosed herein are unique, non-pharmacological interventions for a number of diseases and conditions, including obesity (e.g., diet-induced obesity), diabetes, and other related medical conditions, as discussed above. They can, however, also be applied in a prophylactic or preventative manner in order to reduce the risk that a patient will develop one of the diseases or conditions described herein; to reduce the severity of that disease or condition, should it develop; or to delay the onset or progression of the disease or condition. For example, the present methods can be used to treat patients who are of a recommended weight or who are somewhat overweight but are not considered clinically obese. Similarly, the present methods can be used to treat patients who are considered to be at risk for developing diabetes or who are expected to experience a transplant or traumatic injury (e.g., an incision incurred in the course of a surgical procedure).

The physical stimuli delivered to a subject (e.g., a human) can be, for example, vibration(s), magnetic field(s), and ultrasound. The stimuli can be generated with appropriate means known in the art. For example, vibrations can be generated by transducers (e.g., actuators, e.g., electromagnetic actuators), magnetic field can be generated with Helmholtz coils, and ultrasound can be generated with piezoelectric transducers.

The physical stimuli, if introduced as mechanical signals (e.g., vibrations), can have a magnitude of at least or about 0.01-10.0 g. In embodiments, physical stimuli may have a magnitude of up to about 4.0 g (e.g., 0.01-4.0 g, inclusive, (e.g., 1 g, 2 g, 3 g, or 4 g)). As demonstrated in the Examples below, signals of low magnitude are effective. Accordingly, the methods described here can be carried out by applying at least or about 0.1-1.0 g (e.g., 0.2-0.5 g, inclusive (e.g., about 0.2 g, 0.25 g, 0.3 g, 0.35 g, 0.4 g, 0.45 g, or 0.50 g)) to the subject. The frequency of the mechanical signal can be at least or about 5-1,000 Hz (e.g., 15 or 20-200 Hz, in embodiments about 30-100 Hz, inclusive (e.g., 30-90 Hz (e.g., 30, 35, 40, 45, 50, or 55 Hz)). For example, the frequency of the mechanical signal can be about 5-100 Hz, in embodiments, about 10-100 Hz, inclusive, (e.g., about 40-90 Hz (e.g., 50, 60, 70, 80, or 90 Hz) or 20-50 Hz (e.g., about 20, 25, 30, 35 or 40 Hz), a combination of frequencies (e.g., a “chirp” signal from 20-50 Hz), as well as a pulse-burst of mechanical information (e.g., a 0.5 s burst of 40 Hz, 0.3 g vibration given at least or about every 1 second during the treatment period). The mechanical signals can be provided on a periodic basis (e.g., once every five to ten minutes, once or twice an hour, once every hour, weekly or daily). The physical signals can last at least or about 0.5 seconds to 200 minutes, in embodiments about 2-60 minutes, inclusive (e.g., 2, 5, 10, 15, 20, 30, 45, or 60 minutes).

The physical signals can be delivered in a variety of ways, including by mechanical means by way of Whole Body Vibration through a ground-based vibrating platform or weight-bearing support of any type. In the case of cells in culture, the culture dish can be placed directly on the platform. Optionally, the platform is incorporated within a cell culture incubator or fermentor so that the signals can be delivered to the cells in order to maintain the temperature and pH of the cell culture medium. For a whole organism, the platform can contacts the subject directly (e.g., through bare feet) or indirectly (e.g., through padding, shoes, or clothing). The platform can essentially stand alone, and the subject can come in contact with it as they would with a bathroom scale (i.e., by simply stepping and standing on an upper surface). The subject can also be positioned on the platform in a variety of other ways. For example, the subject can sit, kneel, or lie on the platform. The platform may bear all of the patient's weight, and the signal can be directed in one or several directions. For example, a patient can stand on a platform vibrating vertically so that the signal is applied in parallel to the long axis of, for example, the patient's tibia, fibula, and femur.

In other configurations, a patient can lie down on a platform vibrating vertically or horizontally. A platform that oscillates in several distinct directions could apply the signal multi-axially, e.g, in a non-longitudinal manner around two or more axes. The platform may include a fastening component for securing the subject thereto. The fastening component may be adjustable and formed of an elastic or inelastic material. The fastening component may be a strap, a band, a tube, a belt, or any other coupling or restraining structure for securing the subject to the platform.

Devices can also deliver the signal focally, using local vibration modalities (e.g., to the subject's abdomen, thighs, or back), as well as be incorporated into other devices, such as exercise devices.

The physical signals can also be delivered by the use of acceleration, allowing a limb, for example, to oscillate back and forth without the need for direct load application, thus simplifying the constraints of local application modalities (e.g., reducing the build-up of fat in limb musculature following joint replacement). As illustrated in FIG. 23, for example, a foot “F” of a subject is secured to a platform “P” by a fastening component “C”. Foot “F” may include a wound, such as a laceration or diabetic ulcer. The physical signal may be delivered to foot “F” by vibrations along a single axis “A”. It should be understood, however, that the physical signals may be applied along more than one axis, as discussed above. The physical signals may be transmitted to the platform from a separate device (not shown), or from a device that is integral with (e.g., housed in) the platform.

As discussed above, providing low-magnitude, high-frequency mechanical signals can be done by placing the subject on a device with a vibrating platform. An example of a device is the JUVENT 1000 (by Juvent, Inc., Somerset, N.J.) (see also U.S. Pat. No. 5,273,028). The source of the mechanical signal (e.g., a platform with a transducer, e.g., an actuator, and source of an input signal, e.g., electrical signal) can be variously housed or situated (e.g., under or within a chair, bed, exercise equipment, mat (e.g., a mat used to exercise (e.g., a yoga mat)), hand-held or portable device, a standing frame or the like). The source of the mechanical signal (e.g., a platform with a transducer, e.g., an actuator and a source of an input signal, e.g., electrical signal) can also be within or beneath a floor or other area where people tend to stand (e.g., a floor in front of a sink, stove, window, cashier's desk, or art installation or on a platform for public transportation) or sit (e.g., a seat in a vehicle (e.g., a car, train, bus, or plane) or wheelchair). The signal can also be introduced through oscillatory acceleration in the absence of weightbearing (e.g., oscillation of a limb), using the same frequencies and accelerations as described above.

Electromagnetic field signals can be generated via Helmholtz coils, in the same frequency range as described above, and within the intensity range of 0.1 to 1000 micro-Volts per centimeter squared. Ultrasound signals can be generated via piezoelectric transducers, with a carrier wave in the frequency range described herein, and within the intensity range of 0.5 to 500 milli-Watts per centimeter squared. Ultrasound can also be used to generate vibrations described herein.

The transmissibility (or translation) of signals through the body is high, therefore, signals originating at the source, e.g., a platform with a transducer and a source of, e.g., electrical, signal, can reach various parts of the body. For example, if the subject stands on the source of the physical signal, e.g., the platform described herein, the signal can be transmitted through the subject's feet and into upper parts of the body, e.g., abdomen, shoulders etc.

As described in the Examples below, high frequency, low magnitude mechanical signals were delivered to mice via whole body vibration. When considering the potential to translate this to the clinic, it is important to note that associations persist between vibration and adverse health conditions, including low-back pain, circulatory disorders and neurovestibular dysfunction (Magnusson et al., Spine 21:710-17, 1996), leading to International Safety Organization advisories to limit human exposure to these mechanical signals (International Standards Organization. Evaluation of Human Exposure to Whole-Body Vibration. ISO 2631/1. 1985. Geneva). At the frequency (90 Hz) and amplitude used in the studies described herein (0.4 g peak-to-peak), the exposure would be considered safe for over four hours each day.

### EXAMPLES

### Example 1

### Biomechanical Treatment Improves Glucose Tolerance and Reduces Fat Content in Mice Prone to Obesity

C3H.B6-6T mice, bred as a congenic strain, have reduced (about 20%) circulating IGF-1 (insulin-like growth factor-1) and are phenotypically prone to obesity, despite being smaller than B6 mice. The congenic mice have reduced (by approximately 20%) circulating IGF-1 (C3H.B6-6T [6T]) and were generated by backcrossing a small genomic region (30 cM) of chromosome 6 (Chr6) from C3H/HeJ (C3H) onto a C57B1/6J (B6) background. Thus, they are a unique strain, a “cross” of B6 and C3H.

Half of the C3H.B6-6T seven-week old female mice used in the study were treated by applying a mechanical signal at 0.2 g, 90 Hz for 15 min/day, while the other, untreated mice were used as controls. The five-days-per-week protocol was carried out for 9 weeks with the animals sacrificed at 16 weeks of age. Glucose tolerance was analyzed at eight weeks. Fat content of the thoracic cavity was determined two days before euthanasia by in vivo high-resolution micro-computed tomography scans (In Viva CT, Scanco, Inc.). Triglycerides (TG) and free fatty acid (FFA) were measured by extracting lipid from the serum, adipose tissue (peripheral/visceral), liver and the soleus muscle.

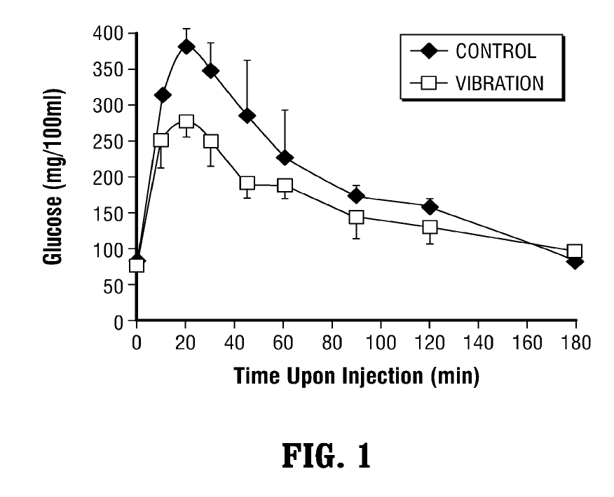

Glucose tolerance in the vibrated animals (analyzed at eight weeks) showed marked improvement in tolerance to insulin, as compared to controls (see FIG. 1).

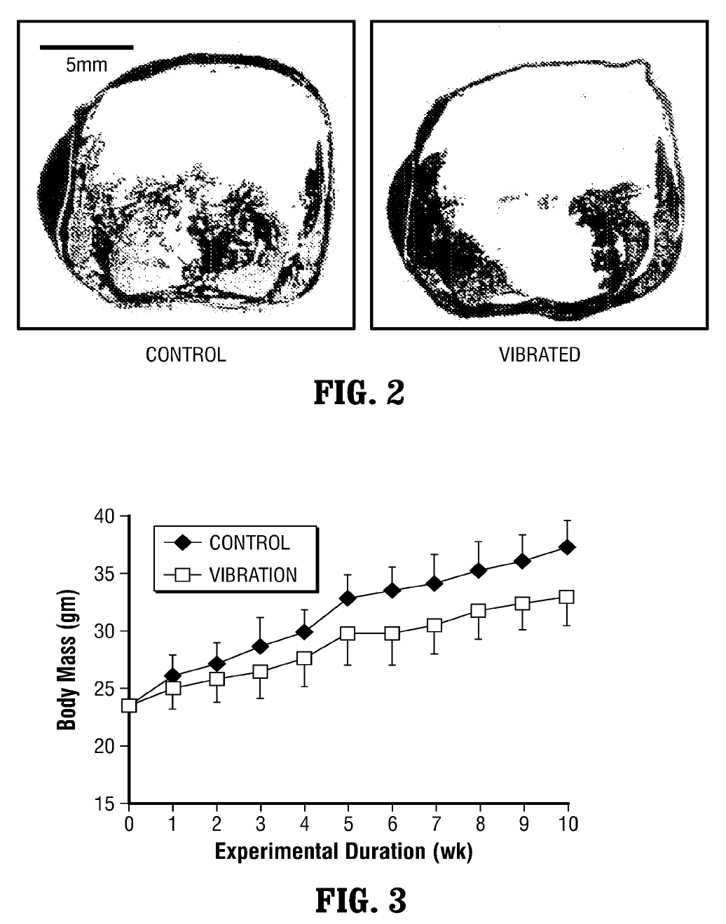

The in vivo scans of the thorax showed that the experimental animals had approximately 18% less volume of visceral fat than the controls (see FIG. 2).

Fasting glucose and insulin levels were unchanged between treated and control groups, suggesting that there was no significant effect on liver or beta cell function. The treated animals showed a 28% reduction in serum free fatty acids when compared to the controls. In the soleus muscle, the treated group showed 13% reduction in triglycerides and a 45% reduction in free fatty acids. In the adipose tissue, the vibrated group showed a 41% reduction in triglycerides and a 47% reduction in free fatty acids.

### Example 2

### Biomechanical Treatment Suppresses the Gain of Body Mass in Normal Mice Fed a High-Fat Diet and Normal Diet

In a follow-up study using “normal” mice, 10-week-old C57BL/6J male mice (n=40) were fed a high-fat diet and treated by exposure to mechanical signals for a brief period each day. The treatment was carried out at 0.2 g, 90 Hz, as in Example 1. These mice showed a markedly lower body mass three weeks into the study than the controls (p<0.05 for all the remaining weeks), reaching a 13% difference at 10 weeks, despite identical food intake (see FIG. 3). At this point, total fat, summed for the entire torso, was 26% lower in the treated animals (p<0.007).

Vibrated mice fed a normal-fat diet were 8% lighter than controls at 10 weeks (p<0.05) and had 15% less body fat. Triglyceride and FFA levels were significantly reduced in the liver, adipose, and muscle tissues of these animals.

These data suggest that these biomechanical signals improve glucose tolerance and even reduce visceral fat content, indicating a unique, and perhaps interrelated, means of controlling long-term consequences of diabetes and obesity.

### Example 3

### Biomechanical Treatment Suppresses the Gain of Body Mass and Fat Content of Normal Mice Fed a Normal Diet

In one experiment, forty C57BL/6J male mice, 7 weeks old and fed a normal diet, were randomly separated into either a mechanically stimulated (MS) or control (CO) group. For 14 weeks, five days per week, the MS mice were subject to 15 minutes per day of a 90 Hz, 0.2 g whole body vibration induced via a vertically oscillating platform. A mechanical vibration at this magnitude and frequency is barely perceptible to human touch. Upon 12 weeks on their respective protocols (19 weeks of age), in vivo micro-CT scans were used to quantify subcutaneous and visceral fat of the torso (n=12 in each group). At sacrifice (21 weeks of age), weights of epididymal fat pad, subcutaneous fat pad, liver and heart were analyzed (all animals).

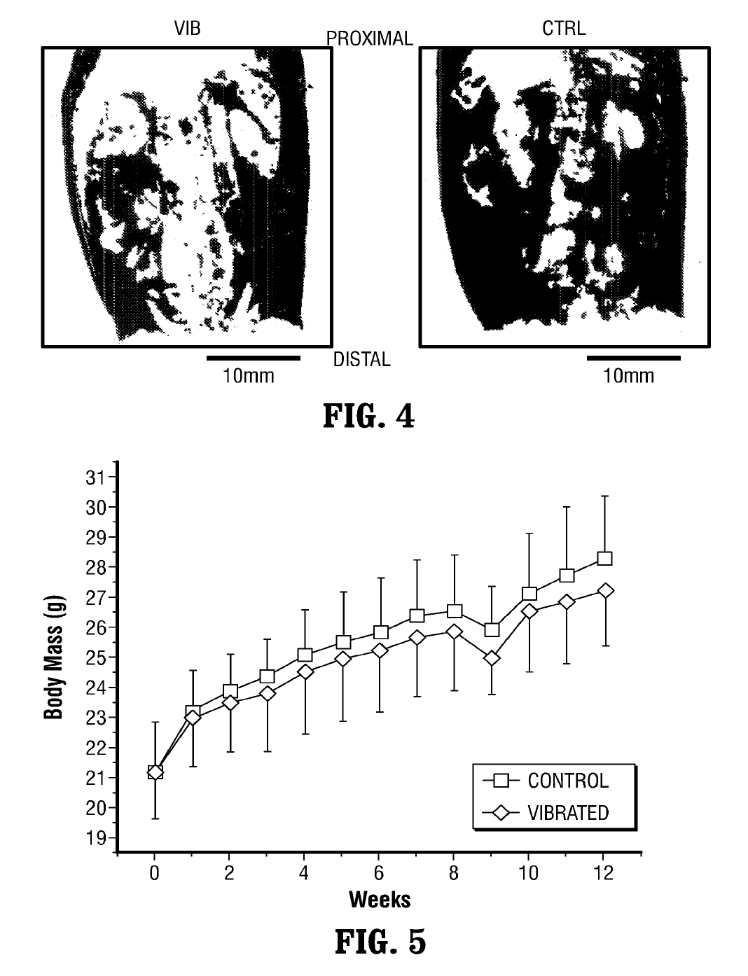

Following a 14 week exposure to short-duration, low-level whole body vibrations, food intake was 7.9% lower, and body mass was 6.7% lower as compared to control mice (p<0.05). In vivo CT measures indicated fat volume in the torso of the MS was 27.6% lower as compared to CO (p<0.005) (see FIG. 4). CT measures were directly supported by the weights of the dissected fat pads, where MS had 22.5% less epididymal and 19.5% less subcutaneous fat than CO (p<0.01). No difference in bone length or heart and liver weights was detected between the groups.

In yet another experiment, forty C57BL/6J male mice, seven weeks of age and fed ad libitum a normal rat chow diet, were randomly separated into one of two groups: those subjected to brief periods of whole body vibrations (WBV; n=20) or their age-matched sham controls (CTR; n=20). All procedures were reviewed and approved by the university's animal use committee. Animal weights, as well as their individual food consumption, were measured on a weekly basis. For fifteen weeks, five days per week, WBV mice were subject to fifteen minutes per day of a 90 Hz, 0.4 g peak-to-peak acceleration (1 g=earth's gravitational field, or 9.8 m·s²), induced by vertical whole body vibration via a closed-loop feedback controlled, oscillating platform (modified DMT plate from Juvent, Inc, NJ) (Fritton et al., Ann. Biomed. Eng. 25:831-39, 1997). A sinusoidal vibration at this magnitude and frequency causes a displacement of approximately 12 microns and is barely perceptible to human touch. CTR animals were also placed on the vibrating platform each day, but the plate was not activated.

Twelve weeks into the protocol (animals at 19 w of age), in vivo micro-computed tomographic scans (VivaCT 40, Scanco Inc, SUI) were used to quantify fat and lean volume of the torso (n=15 in each group). The entire torso of each mouse was scanned at an isotropic voxel sixe of 76 microns (45 kV, 133 μA, 300 ms integration time), and noise was removed from the images with a Gaussian filter (sigma=1.5, support=3.0). The length of the torso was defined by two precise anatomical landmarks, one at the base of the pelvis and the other at the base of the neck. Image segmentation was calibrated using the density range of a freshly harvested fat pad from a B6 mouse unrelated to this study.

At 15 w into the protocol (22 w of age), eight mice from each group were fasted for 14-16 h prior to blood collection. Samples were collected by cardiac puncture with the animal under anaesthesia and the plasma separated by centrifugation (14,000 rpm, 15 min, 4° C.) and kept frozen until analysis. All mice were then killed by cervical dislocation and the different tissues (epididymal fat pad, subcutaneous fat pad, liver, and heart) quickly excised, weighed, frozen in liquid nitrogen and stored at −80° C. for further analyses.

Glycerol and insulin were measured in the plasma, and triglycerides (TG) and non-esterified free fatty acids (NEFA) were measured by extracting lipid from adipose tissue (n=8 per group) and liver (n=12 per group). Plasma insulin levels were measured using an ELISA kit (Mercodia Inc., Winston-Salem, N.C.). TG and NEFA from plasma and tissues were measured using enzymatic calorimetric kits: Serum Triglyceride Determination Kit (Sigma, Saint Louis, Mo.) and NEFA C (Wako Chemicals, Richmond, Va.), respectively. Total lipids from white adipose tissue (epididymal fat pad) and liver were extracted and purified following the chloroform-methanol method (Folch et al., J. Biol. Chem. 226:497-509, 1957) with some modifications, while liver glycogen content were determined by the anthrone method (Seifter et al., Arch. Biochem. 25:191-200, 950).

At baseline, body weights of WBV (21.1 g±1.7 g) and CTR (21.2 g±1.5) were similar (0.25% lower in WBV; p=0.9). Throughout the course of the protocol, weekly food intake between WBV (26.4 gw⁻¹±2.1) and CTR (27.0 gw⁻¹±2.1) was also similar (2.3% lower in WBV, p=0.3). Activity patterns during the fifteen minutes of sham (CTR) or vibration (WBV) treatment were not noticeably different from their behavior in their cages, or from each other. At 12 w, when the in vivo CT scans were performed, the body mass of WBV animals was not significantly different from CTR (4.0% lower in WBV, p=0.2; FIG. 5).

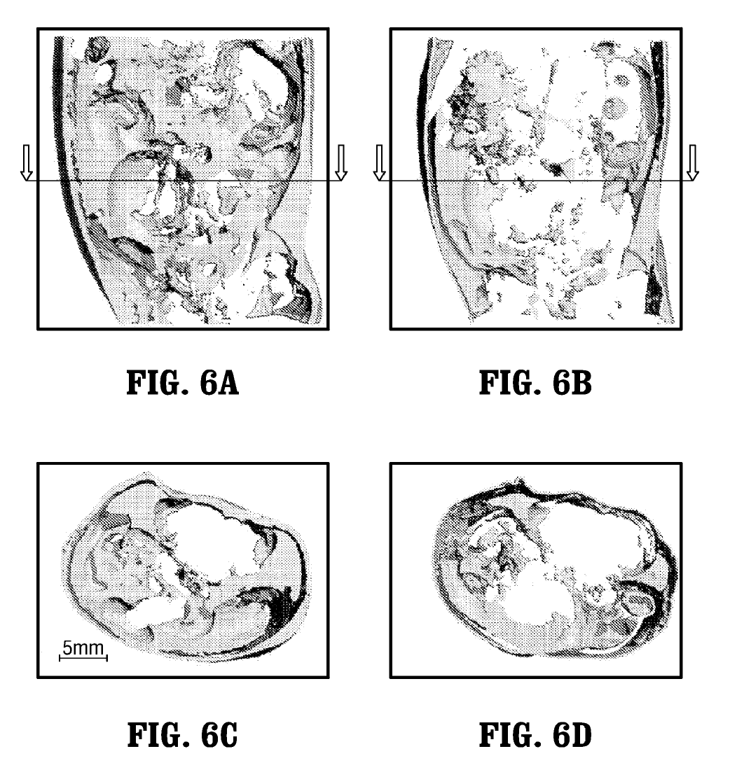

As measured at 12 w by in vivo CT, fat volume in the torso of WBV mice was 25.6% lower than that measured in CTR mice (p=0.01; FIGS. 6A-6D). In contrast, total lean volume of the torso was similar between WBV and CTR (p=0.7; Table 1 below), while lean volume as a ratio of body mass was 4.9% greater in WBV than CTR (p=0.01). Bone volume of the skeleton, from base of the skull to the distal region of the tibia, as a ratio of body mass was 5.9% greater in WBV than CTR (p=0.02). Fat volume normalized to body mass was 21.7% less in the WBV compared to controls (p=0.008). No differences in femoral length (p=0.6), the length of the torso (p=0.6), lean volume (p=0.5), heart (p=0.7) or liver weights (p=0.6), were measured between groups.

TABLE 1
Mean and standard deviation, as well as percentage difference and p-
values, of body habitus parameters at week 12 of the Control and
Vibrated mice, as defined by in vivo microcomputed tomography
(n = 15 in each group, p-values <0.05 are in bold).

| PARAMETERS | CONTROL | VIBRATED | % DIFF | P |
|---|---|---|---|---|
| Body Mass @ 12 | 28.6 ± 2.49 | 27.4 ± 2.21 | −4.0 | 0.20 |
| weeks (g) | | | | |
| Fat Volume (cm³) | 5.33 ± 1.67 | 3.96 ± 0.95 | −25.6 | 0.012 |
| Bone Volume (cm³) | 0.59 ± 0.07 | 0.60 ± 0.08 | +1.9 | 0.701 |
| Lean Volume (cm³) | 18.1 ± 1.3 | 18.3 ± 1.6 | +1.0 | 0.740 |
| Fat Volume/Body | 0.18 ± 0.04 | 0.14 ± 0.03 | −21.7 | 0.008 |
| Mass (cm³/g) | | | | |
| Bone Volume/Body | 0.021 ± 0.001 | 0.022 ± 0.001 | +5.9 | 0.024 |
| Mass (cm³/g) | | | | |
| Lean Volume/Body | 0.64 ± 0.03 | 0.67 ± 0.03 | +4.9 | 0.010 |
| Mass (cm³/g) | | | | |
| Skeletal Length (cm) | 8.17 ± 0.20 | 8.21 ± 0.17 | +0.5 | 0.580 |
| Fat Volume/Skeletal | 0.65 ± 0.19 | 0.48 ± 0.12 | −25.8 | 0.008 |
| Length (cm²) | | | | |
| Bone Volume/Skeletal | 0.072 ± 0.008 | 0.073 ± 0.009 | +1.4 | 0.743 |
| Length (cm²) | | | | |
| Lean Volume/Skeletal | 2.22 ± 0.13 | 2.23 ± 0.16 | +0.5 | 0.858 |
| Length (cm²) | | | | |
| Fat Mass (g) | 4.90 ± 1.54 | 3.64 ± 0.88 | −25.6 | 0.012 |
| (density = 0.92) | | | | |
| Bone Mass (g) | 1.06 ± 0.13 | 1.08 ± 0.15 | +1.9 | 0.701 |
| (density = 1.80) | | | | |

Fat volume data derived from in vivo CT were supported by the weights of the dissected fat pads performed post-sacrifice at 15 w, where WBV had 26.2% less epididymal (p=0.01) and 20.8% less subcutaneous (p=0.02) fat than CTR (Table 2 below). Normalized to mass, there was 22.5% less epididymal and 19.5% less subcutaneous fat in WBV than CTR (p=0.007).

TABLE 2
Mean and standard deviation, as well as percentage difference and p-
values, of both habitus (n ≧ 15 in each group) and biochemical
parameters (n = 8 in each group), measured directly, post-sacrifice
(n ≧ 15 in each group, p-values < 0.05 are in bold).

| PARAMETERS | CONTROL | VIBRATED | % DIFF | P |
|---|---|---|---|---|
| Epididymal Fat | 0.63 ± 0.21 | 0.47 ± 0.12 | −26.2 | 0.014 |
| weight (g) | | | | |
| Subcutancous Fat | 0.21 ± 0.06 | 0.17 ± 0.03 | −20.8 | 0.016 |
| weight (g) | | | | |
| Heart weight (g) | 0.120 ± 0.010 | 0.122 ± 0.015 | +1.6 | 0.707 |
| Liver weight (g) | 1.11 ± 0.11 | 1.09 ± 0.09 | −1.7 | 0.581 |
| Plasma Glycerol | 17.37 ± 6.63 | 18.75 ± 9.31 | +7.9 | 0.64 |
| (mg/dL) | | | | |
| Plasma Insulin (ng/mL) | 0.54 ± 0.09 | 0.48 ± 0.07 | −10.8 | 0.068 |
| Plasma TG (mg/dL) | 38.74 ± 15.67 | 39.44 ± 12.4 | +1.8 | 0.89 |
| Plasma FFA (mmol/L) | 0.69 ± 0.32 | 0.63 ± 0.20 | −8.9 | 0.53 |

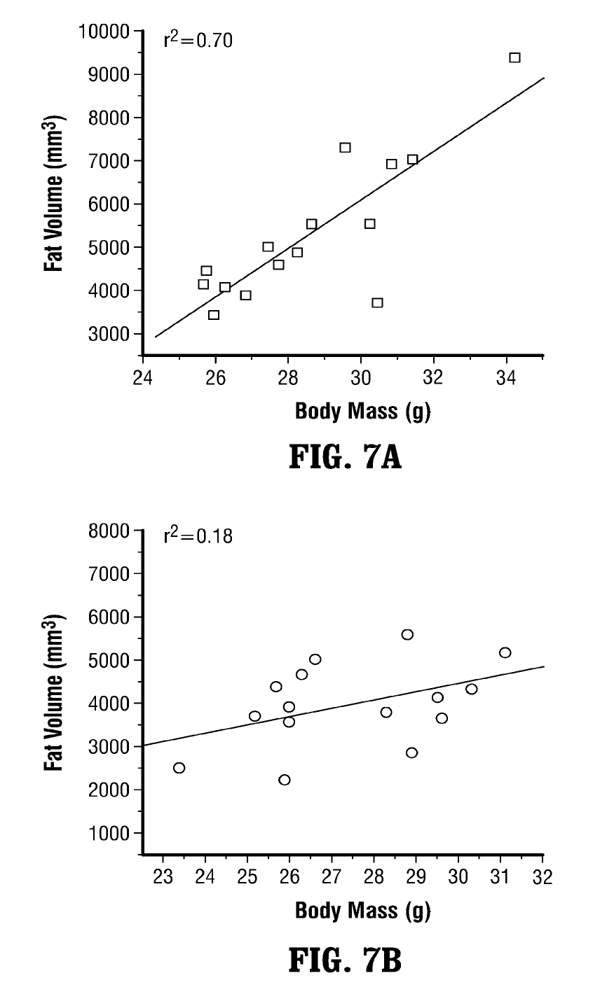

Correlations between food intake and either total body mass (r²=0.15; p=0.7) or fat volume (r²=0.008; p=0.6) were weak, and indicated that the lower adiposity in WBV animals could not be explained by differences in food consumption between the groups. While variations in body mass of the CTR mice correlated strongly with fat volume (r²=0.70; p=0.0001), no such correlation was observed in WBV (r²=0.18; p=0.1), indicating that fat mass contributed to weight gain in the controls, but failed to account for the increase in body mass in the mechanically stimulated animals (FIGS. 7A and 7B).

To account for the 1.2 g body mass difference between WBV and CTR mice measured at 12 w, in vivo CT measurements of fat volume were converted to mass equivalents. Using a density of 0.9196 g·cm⁻³ to convert fat volume to fat mass (Watts et al., Metabolism 51:1206-1210, 2002) indicated that the 3.64 g±0.9 of the average WBV mouse mass came from fat (13.3% of total mass), while 4.90 g±1.5 of the mass of the average CTR mouse came from fat (17.1% of total mass). Thus, the lack of fat in the WBV animals was, in essence, able to account for the “missing mass” between the groups (p=0.01).

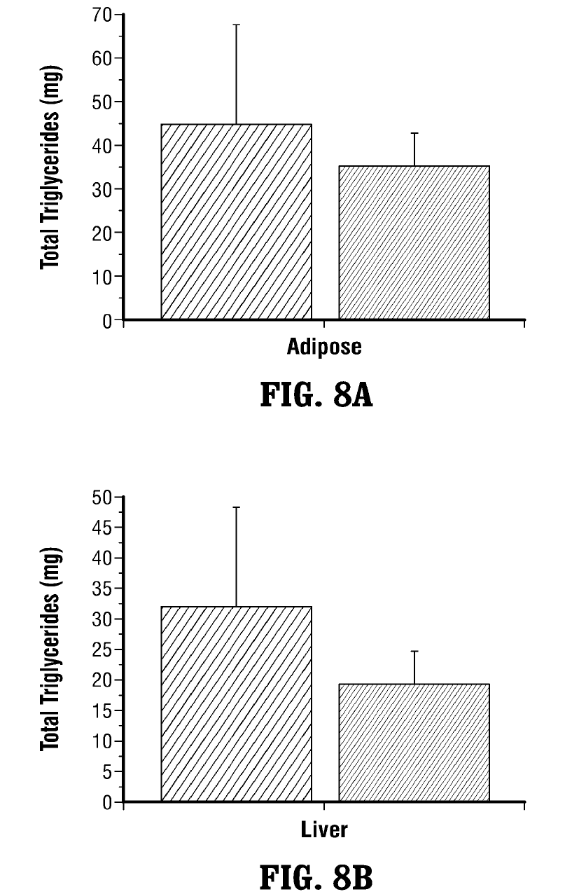

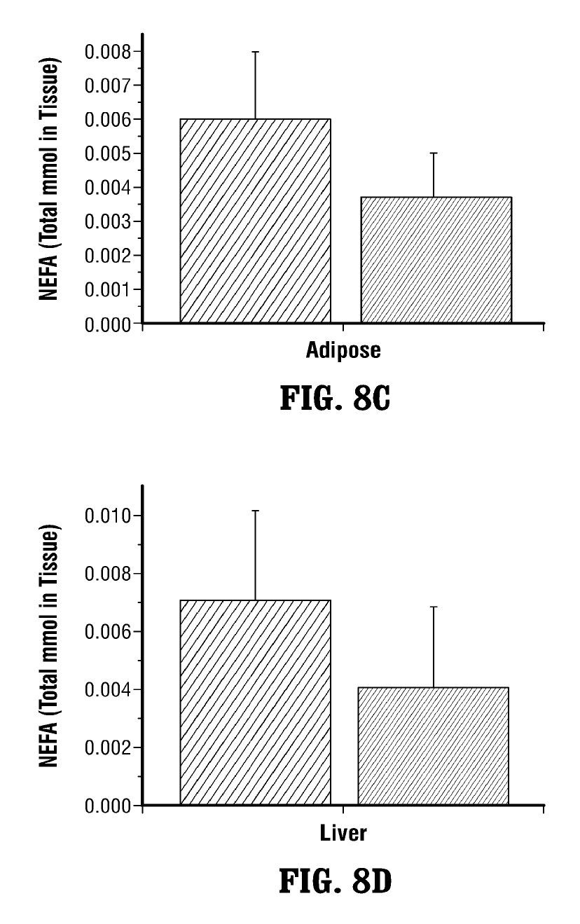

Fasting glucose and insulin levels showed only a trend in decreased plasma insulin in the WBV group (p=0.07), and taken together, these data suggested that these mechanical signals had no significant effect on liver or beta cell function (Table 2 above). At sacrifice, triglycerides (total mg in tissue) in adipose tissue of WBV were 21.1% (p=0.3) lower than CTR, and 39.1% lower in the liver (p=0.02; FIGS. 8A and 8B). Total non-esterified fatty acids (total mmol in tissue) in adipose tissue were 37.2% less in WBV as compared to CTR (p=0.01; FIG. 8C), while NEFA in the liver of WBV (total μmol/mg tissue) mice was 42.6% lower (p=0.02) than CTR (FIG. 8D). Glucose tolerance, tested at 9 w in three animals in each group, was slightly improved in WBV over CTR mice, but this difference was not statistically significant (data not shown).

In contrast to the perception that physical signals must be large and endured over a long period of time to offset caloric input and control insulin production, these results indicate that the cell population(s) and physiologic process(es) responsible for controlling fat mass and free fatty acid and triglyceride production are readily influenced by mechanical signals barely large enough to be perceived, an attribute achieved within an exceedingly short period of time.

The means by which these low-level signals suppress adiposity has been difficult to determine. Certainly, a trend towards improved glucose tolerance indicates that the metabolic machinery of the organism has been elevated, and remains higher long after the subtle challenge of low-level vibration has subsided, suggesting that a mechanosensory element within the cell population can be triggered without the signals necessarily being large (Rubin et al., Gene 367:1-16, 2006). And rather than requiring the accumulation of mechanical information through the product of time and intensity to elevate metabolic activity, perhaps these cell populations and physiologic processes are endowed with a memory, or refractory period, in which their metabolic machinery, once triggered, remains active even after the stimulus has subsided (Skerry et al., J. Orthop. Res. 6:547-551).

These data also suggest that mesenchymal cells are mechanically responsive, and that these physical signals need not be large to influence differentiation pathways. It appears that mesenchymal precursors perceive and respond to these mechanical “demands” as stimuli to differentiate down a musculoskeletal pathway, rather than “defaulting” to adipose tissue.

### Example 4

### Biomechanical Treatment Reduces Severity of Pancreatitis in Pancreatitis Induced Normal Mice Fed a High-Fat Diet

“Normal” C57BL/6 mice were fed a high-fat diet (HFD) (60% kcal from fat) for a total of 13 weeks. After 8 weeks on the HFD, the mice were randomly separated into either a low intensity vibration stimulated (LIV, Non-Inj) group or a control (non-LIV, Non-Inj) group. The LIV, Non-Inj mice were treated with a low intensity vibration at 0.2 g, 90 Hz, for 15 minutes per day, 5 days a week for 5 weeks. After 4 weeks of low intensity vibration treatment, IL-12 and IL-18 were injected into some of the mice treated with low intensity vibration (LIV, IL12+IL18 Inj) and some of the control mice (Non-LIV, IL12+IL18 Inj) to induce pancreatitis (the continued use of the HFD increasing the severity of the pancreatitis). One week after injection of the IL-12 and IL-18 cytokines, all mice were sacrificed and tissues were collected.

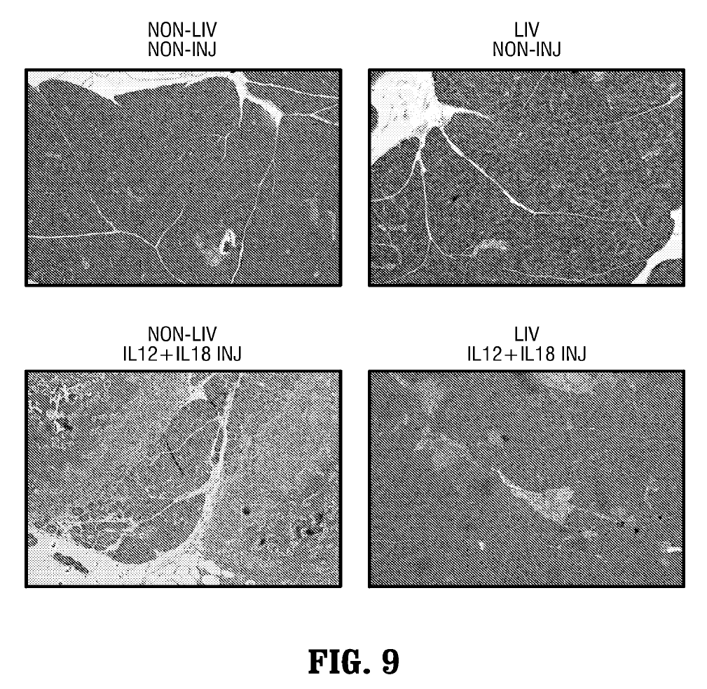

Pancreatic tissue was assessed by histological analysis. The tissue was fixed in formalin, embedded in paraffin, and sections were stained with hematoxylin and eosin. By way of image analysis, no significant difference in appearance of the pancreas was observed between the LIV, Non-Inj mice and the Non-LIV, Non-Inj mice. (Top row of FIG. 9). However, for mice in which pancreatitis was induced, the Non-LIV IL12+IL18 Inj mice showed severe inflammation and tissue damage compared to the LIV, IL12+IL18 Inj mice who showed significantly reduced pathology. (Bottom row of FIG. 9).

These data suggest that the application of low-intensity vibration reduced the severity of pancreatitis disease by reducing inflammation and/or enhancing tissue repair and regeneration to restore the histological appearance of inflamed or damaged tissue towards that seen in the control mice.

Our studies, provided below as examples 5-15, have demonstrated that six weeks of LMMS in C57BL/6J mice can increase the overall marrow-based stem cell population by 37% and the number of MSCs by 46%. Concomitant with the increase in stem cell number, the differentiation postential of MSCs in the bone marrow was biased toward osteoblastic and against adipogenic differentiation, as reflected by upregulation of the transcription factor Runx2 by 72% and downregulation of PPARγ by 27%. The phenotypic impact of LMMS on MSC lineage determination was evident at 14 weeks, where visceral adipose tissue formation was suppressed by 28%.

Accordingly, the present methods employ mechanical signals as a non-invasive means to influence stem cell (e.g., mesenchymal stem cell) or precursor cell proliferation and fate (differentiation). In some instances, that influence will promote bone formation while suppressing the fat phenotype.

### Example 5

### Materials and Methods

Animal Model to Prevent Diet Induced Obesity (DIO). All animal procedures were reviewed and approved by the Stony Brook University animal care and use committee. The overall experimental design consisted of two similar protocols, differing in the duration of treatment to assess mechanistic responses of cells to LMMS (6 w of LMMS compared to control, n=8 per group) or to characterize the phenotypic effects (14 w of LMMS compared to control). Two models of DIO were employed: 1. to examine the ability of LMMS to prevent obesity, a “Fat Diet” condition (n=12 each, LMMS and CON) was evaluated where LMMS and DIO were initiated simultaneously, and 2. to examine the ability of LMMS to reverse obesity, an “Obese” condition (n=8 each, LMMS and CON) was established, whereby LMMS treatment commenced 3 weeks after the induction of DIO, and compared to sham controls.

Mechanical enhancement of stem cell proliferation and differentiation in DIO. Beginning at 7 w of age, C57BL/6J male mice were given free access to a high fat diet (45% kcal fat, #58V8, Research Diet, Richmond, Ind.). The mice were randomized into two groups defined as LMMS (5d/w of 15 min/d of a 90 Hz, 0.2 g mechanical signal, where 1.0 g is earth's gravitational field, or 9.8 m/s2), and placebo sham controls (CON). The LMMS protocol 13 provides low magnitude, high frequency mechanical signals by a vertically oscillating platform, 14 and generates strain levels in bone tissue of less than five microstrain, several orders of magnitude below peak strains generated during strenuous activity. Food consumption was monitored by weekly weighing of food.

Status of MSC pool by flow cytometry. Cellular and molecular changes in the bone marrow resulting from 6 w LMMS (n=8 animals per group, CON or LMMS) were determined at sacrifice from bone marrow harvested from the right tibia and femur (animals at 13 w of age). Red blood cells in the bone marrow aspirate were removed by room temperature incubation with Pharmlyse (BD Bioscience) for 15 mins. Single cell suspensions were prepared in 1% sodium azide in PBS, stained with the appropriate primary and (when indicated) secondary antibodies, and fixed at a final concentration of 1% formalin in PBS. Phycoerythrin (PE) conjugated rat anti-mouse Sca-1 antibody and isotype control were purchased from BD Pharmingen and used at 1:100. Rabbit anti-mouse Pref-1 antibody and FITC conjugated secondary antibody were purchased from Abeam (Cambridge, Mass.) and used at 1:100 dilutions. Flow cytometry data was collected using a Becton Dickinson FACScaliber flow cytometer (San Jose, Calif.).

RNA extraction and real-time RT-PCR. At sacrifice, the left tibia and femur were removed and marrow flushed into an RNAlater solution (Ambion, Foster City, Calif.). Total RNA was harvested from the bone marrow using a modified TRIspin protocol. Briefly, TRIzol reagent (Life Technologies, Gaithersburg, Md.) was added to the total bone marrow cell suspension and the solution homogenized. Phases were separated with chloroform under centrifugation. RNA was precipitated via ethanol addition and applied directly to an RNeasy Total RNA isolation kit (Qiagen, Valencia, Calif.). DNA contamination was removed on column with RNase free DNase. Total RNA was quantified on a Nanodrop spectrophotometer and RNA integrity monitored by agarose electrophoresis. Expression levels of candidate genes was quantified using a real-time RT-PCR cycler (Lightcycler, Roche, Ind.) relative to the expression levels of samples spiked with exogenous cDNA. 15 A “one-step” kit (Qiagen) was used to perform both the reverse transcription and amplification steps in one reaction tube.

qRT-PCR with Content Defined 96 Gene Arrays. PCR arrays were obtained from Bar Harbor Biotech (Bar Harbor, Me.), with each well of a 96 well PCR plate containing gene specific primer pairs. The complete gene list for the osteoporosis array can be found at www.bhbio.com, and include genes that contribute to bone mineral density through bone resorption and formation, genes that have been linked to osteoporosis, as well as biomarkers and gene targets associated with therapeutic treatment of bone loss. cDNA samples were reversed transcribed (Message Sensor RT Kit, Ambion, Foster City, Calif.) from total RNA harvested from bone marrow cells and used as the template for each individual animal. Data were generated using an Applied Biosystems 7900HT real-time PCR machine, and analyzed by Bar Harbor Biotech.

Body habitus established by in vivo microcomputed tomography (μCT). Phenotypic effects of DIO, for both the “prevention” and “reversal” of obesity test conditions were defined after 12 and 14 w of LMMS. At 12 w, in vivo μCT scans were used to establish fat, lean, and bone volume of the torso (VivaCT 40, Scanco Medical, Bassersdorf, Switzerland). Scan data was collected at an isotropic voxel size of 76 μm (45 kV, 133 μA, 300-ms integration time), and analyzed from the base of the skull to the distal tibia for each animal. Threshold parameters were defined during analysis to segregate and quantify fat and bone volumes. Lean volume was defined as animal volume that is neither fat nor bone, and includes muscle and organ compartments.

Bone phenotype established by ex vivo microcomputed tomography. Trabecular bone morphology of the proximal region of the left tibia of each mouse was established by μCT at 12 μm resolution (μCT 40, Scanco Medical, Bassersdorf, Switzerland). The metaphyseal region spanned 600 μm, beginning 300 μm distal to the growth plate. Bone volume fraction (BV/TV), connectivity density (Conn.D), trabecular number (Tb.N), trabecular thickness (Tb.Th), trabecular separation (Tb.Sp), and the structural model index (SMI) were determined.

Serum and tissue biochemistry. Blood collection was performed after overnight fast by cardiac puncture with the animal under deep anesthesia. Serum was harvested by centrifugation (14,000 rpm, 15 min, 4° C.). Mice were euthanized by cervical dislocation, and the different tissues (i.e., epididymal fat pad and subcutaneous fat pads from the lower torso, liver, and heart) were excised, weighed, frozen in liquid nitrogen, and stored at −80° C. Total lipids from white adipose tissue (epididymal fat pad) and liver were extracted and purified based on a chloroform-methanol extraction. Total triglycerides (TG) and non-esterified free fatty acids (NEFA) were measured on serum (n=10 per group) and lipid extracts from adipose tissue (n=5 or 6 per group) and liver (n=10 per group) using enzymatic colorimetric kits (TG Kit from Sigma, Saint Louis, Mo.; and NEFA C from Wako Chemicals, Richmond, Va.). ELISA assays were utilized to determine serum concentrations of leptin, adiponectin, resistin (all from Millipore, Chicago, Ill.), osteopontin (R&D Systems, Minneapolis, Minn.), and osteocalcin (Biomedical Technologies Inc, Stoughton, Mass.), using a sample size of n=10 per group.

Human pilot trial to examine inverse relationship of adipogenesis and osteoblastogenesis. A trial designed and conducted to evaluate if 12 months of LMMS could promote bone density in the spine and hip of women with low bone density was evaluated retrospectively to examine changes in visceral fat volume. All procedures were reviewed and approved by the Childrens Hospital of Los Angeles Committee on Research in Human Subjects.

Forty-eight healthy young women (aged 15-20 years) were randomly assigned into either LMMS or CON groups (n=24 in each group). The LMMS group underwent brief (10 min requested), daily treatment with LMMS (30 Hz signal @ 0.3 g) for one year. Computed tomographic scans (CT) were performed at baseline and one year, with the same scanner (model CT-T 9800, General Electric Co., Milwaukee, Wis.), the same reference phantom for simultaneous calibration, and specially designed software for fat and muscle measurements. Identification of the abdominal site to be scanned was performed with a lateral scout view, followed by a cross-sectional image obtained from the midportion of the third lumbar vertebrae at 80 kVp, 70 milliamperes, and 2S.

Cancellous bone of the 1st, 2nd and 3rd lumbar vertebrae was established as measures of the tissue density of bone in milligrams per cubic centimeter (mg/cm3). Area of visceral fat (cm2) was defined at the midportion of the third lumbar vertebrae as the intra-abdominal adipose tissue surrounded by the rectus abdominus muscles, the external oblique muscles, the quadratus lumborum, the psoas muscles and the lumbar spine at the midportions of the third lumbar vertebrae, and consisted mainly of perirenal, pararenal, retroperitoneal and mesenteric fat. The average area of paraspinous musculature (cm2) was defined as the sums of the area of the erector spinae muscles, psoas major muscles and quadratus lumborum muscles at the midportion of the third lumbar vertebrae. 18 All analyses of bone density, and muscle and fat area were performed by an operator blinded as to subject enrollment.

Statistical analyses. All data are shown as mean±standard deviation, unless noted. To determine significant differences between LMMS and CON groups, two tailed t-tests (significance value set at 5%) were used throughout. Animal outliers were determined based on animal weight at baseline (before the start of any treatment) as animals falling outside of two standard deviations from the total population, or in each respective group at the end of 6 or 14 weeks LMMS (or sham CON) by failure of the Weisberg one-tailed t-test (alpha=0.01), regarded as an objective tool for showing consistency within small data sets. 19 No outliers were identified in the 6 w CON and LMMS groups. Two outliers per group (CON and LMMS) were identified in the Fat Diet model (14 w LMMS study) and removed. Data from these animals were not included in any analyses, resulting in a sample size of n=10 per group for all data, unless otherwise noted. No outliers were identified in the 14 w Obese model (n=8). Data presented from the human trial are based on the intent to treat data set (all subjects included in the evaluation). Changes in visceral fat volume were compared between LMMS and CON subjects using a one tailed t-test.

### Example 6

### Bone Marrow Stem Cell Population is Promoted by LMMS

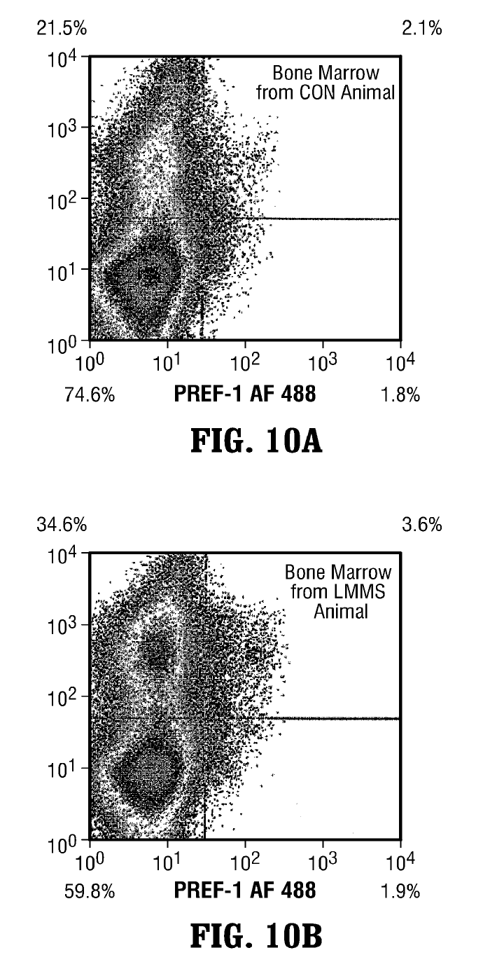

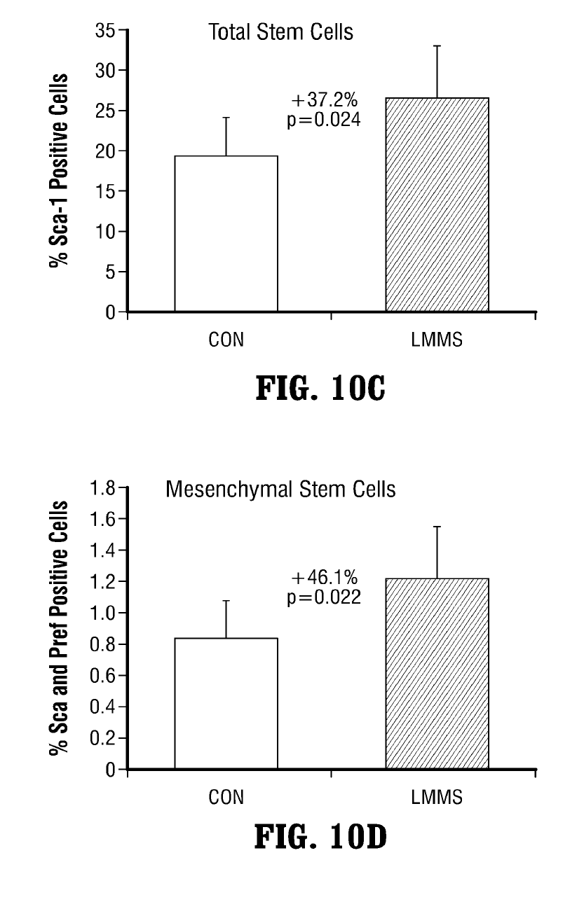

Flow cytometric measurements using antibodies against Stem Cell Antigen-1 (Sca-1) indicated that in animals in the “prevention” DIO group, 6 w of LMMS treatment significantly increased the overall stem cell population relative to controls, as defined by cells expressing Sca-1. Analysis focused on the primitive population of cells with low forward (FSC) and side scatter (SSC), indicating the highest Sca-1 staining for all cell populations. Cells in this region demonstrated a 37.2% (p=0.028) increase in LMMS stem cell numbers relative to sham CON animals. Mesenchymal stem cells as represented by cells positive for Sca-1 and Preadipocyte Factor-1 (Pref-1), 1 represented a much smaller percentage of the total cells. Identified in this manner, in addition to the increase in the overall stem cell component, LMMS treated animals had a 46.1% (p=0.022) increase in mesenchymal stem cells relative to CON (FIG. 10).

### Example 7

### LMMS Biases Marrow Environment and Lineage Commitment

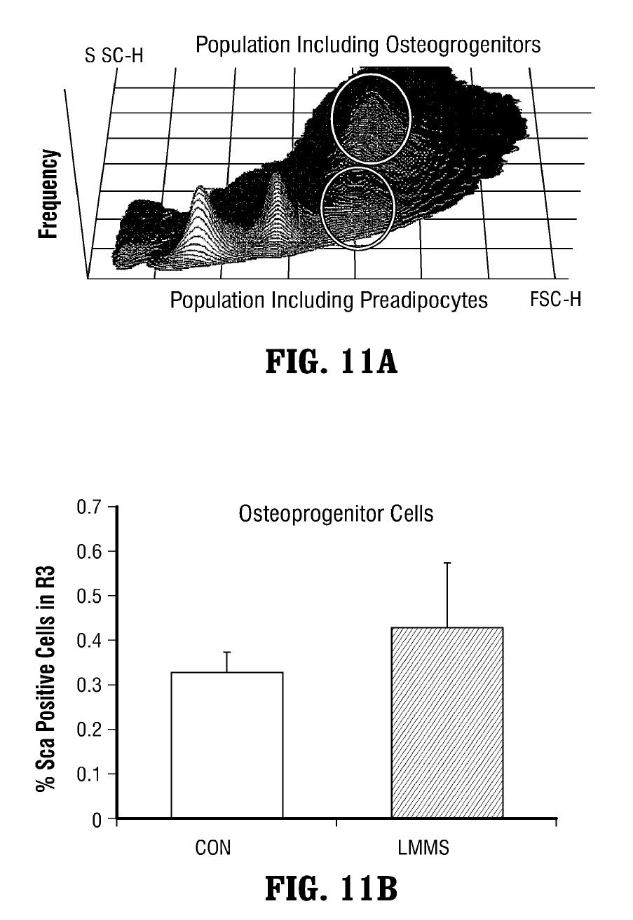

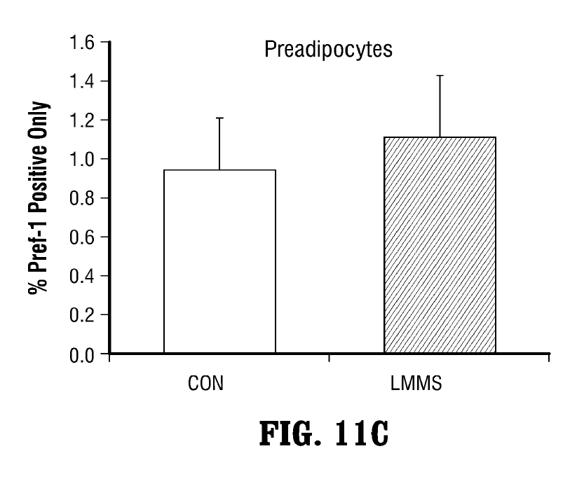

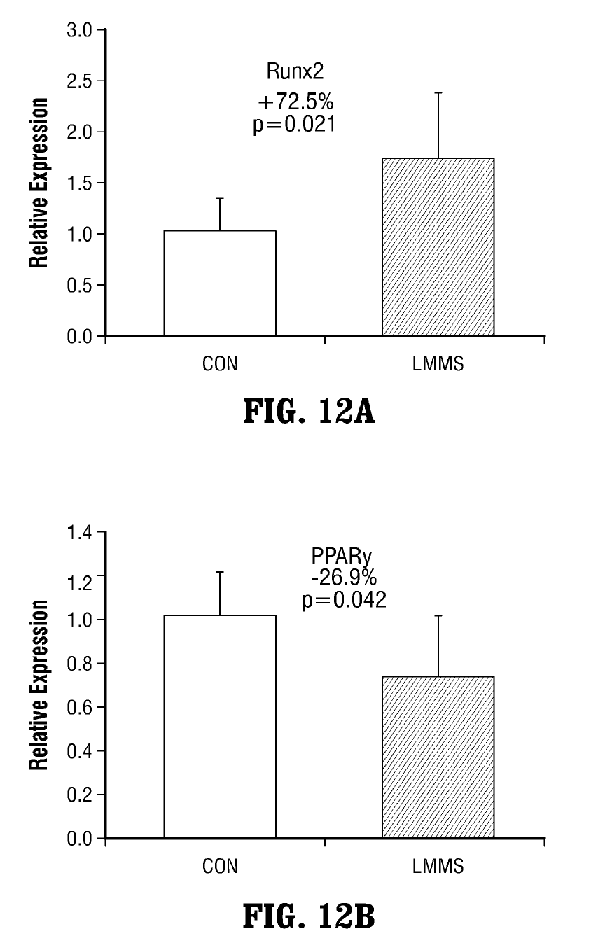

After six weeks, cells expressing only the Pref-1 label, considered committed preadipocytes, were elevated by 18.5% (p=0.25) in LMMS treated animals relative to CON (FIG. 11). Osteoprogenitor cells in the bone marrow population, identified as Sca-1 positive with high FSC and SSC, 20 were 29.9% greater (p=0.23) greater when subject to LMMS. This trend indicating that differentiation in the marrow space of LMMS animals had shifted towards osteogenesis was confirmed by gene expression data, which demonstrated that transcription of Runx2 in total bone marrow isolated from LMMS animals was upregulated 72.5% (p=0.021) relative to CON. In these same LMMS animals, expression of PPARγ was downregulated by 26.9% (p=0.042) relative to CON (FIG. 12).

Gene expression data on bone marrow samples were also tested on a 96 gene “osteoporosis” array, which included genes that contribute to bone mineral density through bone resorption and formation, and genes that have been linked to osteoporosis through association studies. Samples for both CON and LMMS groups expressed 83 of the 94 genes present on the array. qRT-PCR arrays reported decreases in genes such as Pon1 (paraoxonase-1), is known to be associated with high density lipoproteins (−137%, p=0.263), and sclerostin (−258%, p=0.042), which antagonizes bone formation by acting on Wnt signaling. 21 Genes such as estrogen related receptor (Esrra; +107%, p=0.018) and Pomc-1 (pro-opiomelanocortin, +68%, p=0.055) were up-regulated by LMMS.

### Example 8

### LMMS Enhancement of Bone Quantity and Quality

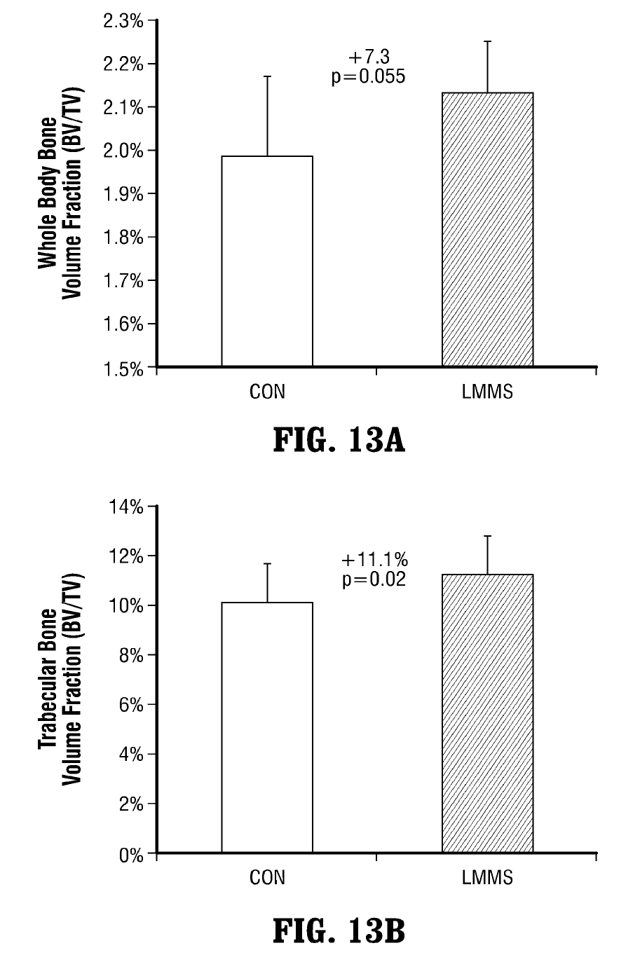

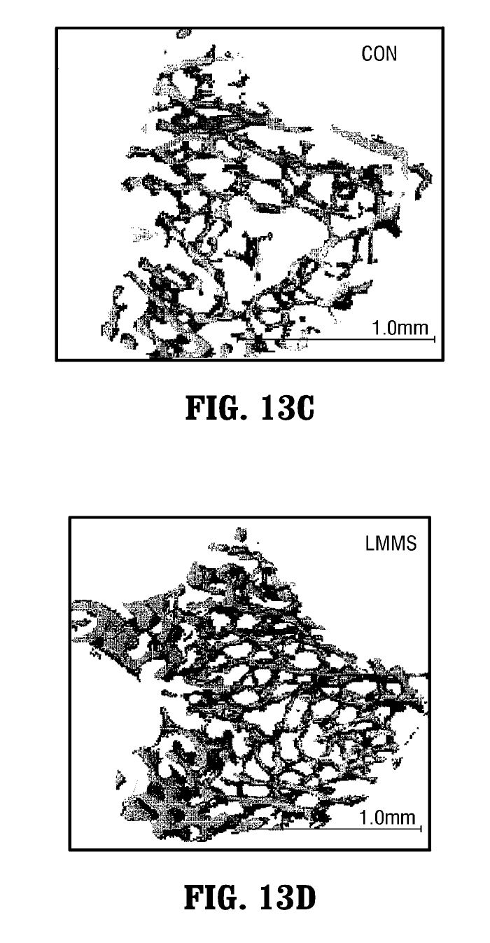

The ability of LMMS induced changes in proliferation and differentiation of MSCs to elicit phenotypic changes in the skeleton was first measured at 12 w by in vivo μCT scanning of the whole mouse (neck to distal tibia). Animals subject to LMMS showed a 7.3% (p=0.055) increase in bone volume fraction of the axial and appendicular skeleton (BV/TV) over sham CON. Post-sacrifice, 12 μm resolution μCT scans of the isolated proximal tibia of the LMMS animals showed 11.1% (p=0.024) greater bone volume fraction than CON (FIG. 13). The micro architectural properties were also enhanced in LMMS as compared to CON, as evidenced by 23.7% greater connectivity density (p=0.037), 10.4% higher trabecular number (p=0.022), 11.1% smaller separation of trabeculae (p=0.017) and a 4.9% lower structural model index (SMI, p=0.021; Table 3 below).

TABLE 3
Micro-architectural parameters of trabecular bone in fat diet
animals measured at 14 w (mean ± s.d., n = 10) demonstrate the
enhanced structural quality of bone in the proximal tibia of LMMS
treated animals as compared to controls

| | CON | LMMS | % diff | p-value |
|---|---|---|---|---|
| Conn. D | 105.3 ± 34.2 | 130.3 ± 28.9 | 23.7 | 0.037 |
| (1/mm³) | | | | |
| Tb. N | 3.06 ± 0.45 | 3.38 ± 0.37 | 10.4 | 0.022 |
| (1/mm) | | | | |
| Tb. Th | 0.029 ± 0.001 | 0.030 ± 0.001 | 1.0 | 0.398 |
| (mm) | | | | |
| Tb. Sp | 0.304 ± 0.046 | 0.270 ± 0.035 | −11.1 | 0.017 |
| (mm) | | | | |
| SMI | 2.93 ± 0.22 | 2.78 ± 0.14 | −4.9 | 0.021 |

### Example 9

### Prevention of Obesity by LMMS

At 12 w, neither body mass gains nor the average weekly food intake differed significantly between the LMMS or CON groups (Table 4 below). At this point (19 wks of age), CON weighed 32.9 g±4.2 g, while LMMS mice were 6.8% lighter at 30.7 g±2.1 g (p=0.15). CON were 15.0% heavier than mice of the same strain, gender and age that were fed a regular chow diet, 13 and increase in body mass due to high fat feeding was comparable to previously reported values. 22 Adipose volume from the abdominal region (defined as the area encompassing the lumbar spine) was segregated as either subcutaneous or visceral adipose tissue (SAT or VAT, respectively). LMMS animals had 28.5% (p=0.021) less VAT by volume, and 19.0% (p=0.016) less SAT by calculated volume. Weights of epididymal fat pads harvested at sacrifice (14 w) correlated strongly with fat volume data obtained by CT. The epididymal fat pad weight was 24.5% (p=0.032) less in LMMS than CON, while the subcutaneous fat pad at the lower back region was 26.1% (p=0.018) lower in LMMS (Table 4 below).

TABLE 4
Despite similar body mass and weekly food consumption,
phenotypic parameters of the fat diet animals after 12 w of LMMS
or at sacrifice (14 w, mean ± s.d., n = 10) demonstrate a leaner
body habitus, as the adipose burden (visceral and subcutaneous
fat) is significantly lower in the LMMS animals.

| | CON | LMMS | % diff | p-value |
|---|---|---|---|---|
| Animal Weight at 12 weeks | 32.9 ± | 30.7 ± 2.74 | −6.8 | 0.152 |
| (grams) | 4.12 | | | |
| Weekly Food Consumption | 18.9 ± | 18.5 ± 1.47 | −2.5 | 0.406 |
| (grams) | 1.67 | | | |
| Visceral Adipose Tissue | 2.3 ± | 1.6 ± 0.34 | −28.5 | 0.021 |
| (VAT, cm³) | 0.72 | | | |
| Subcutaneous Adipose Tissue | 0.84 ± | 0.68 ± 0.08 | −19.0 | 0.016 |
| (SAT, cm³) | 0.16 | | | |
| Epididymal Fat Pad | 1.85 ± | 1.40 ± 0.32 | −24.5 | 0.032 |
| (grams) | 0.52 | | | |
| Subcutaneous Fat Pad | 0.67 ± | 0.50 ± 0.12 | −26.1 | 0.018 |
| (grams) | 0.17 | | | |
| Liver | 0.99 ± | 0.94 ± 0.07 | −4.9 | 0.399 |
| (grams) | 0.16 | | | |

### Example 10

### LMMS Prevents Increased Biochemical Indices of Obesity

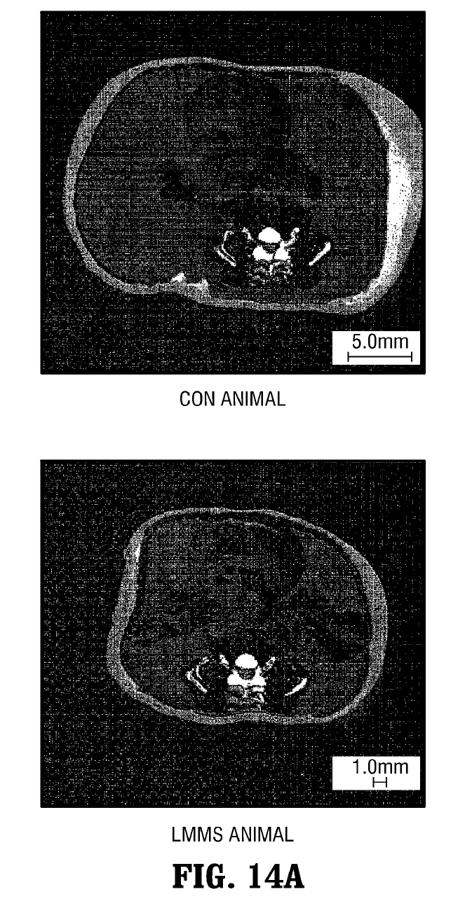

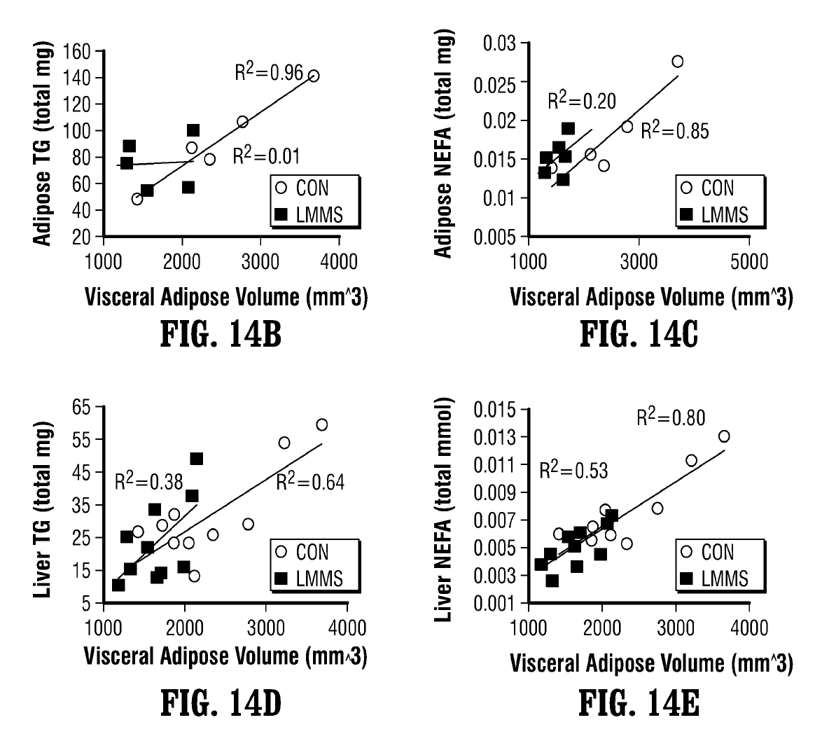

Triglycerides (TG) and non-esterified free fatty acids (NEFA) measured in plasma, epididymal adipose tissue, and liver were all lower in LMMS as compared to CON (Table 5 below). Liver TG levels decreased by 25.6% (p=0.19) in LMMS animals, paralleled by a 33.0% (p=0.022) decrease in NEFA levels. Linear regressions of adipose and liver TG and NEFA values to μCT visceral volume (VAT) demonstrated strong positive correlations for CON animals, with R2=0.96 (p=0.002) for adipose TG, R2=0.85 (p=0.027) for adipose NEFA, R2=0.64 (p=0.006) for liver TG and R2=0.80 (p=0.003) for liver NEFA (FIG. 14). LMMS resulted in weaker correlations between all TG and NEFA levels to increases in VAT.

TABLE 5
Biochemical parameters of the fat diet animals (mean ± s.d.,
n = 10) highlight lower level of TG, NEFA, and circulating adipokines
following 14 w of LMMS stimulation as compared to controls.

| | CON | LMMS | % diff | p-value |
|---|---|---|---|---|
| TG Liver | 31.8 ± 14.3 | 23.6 ± 12.7 | −25.6 | 0.195 |
| (total mg) | | | | |
| NEFA Liver | 7.5 ± 2.7 | 5.0 ± 1.5 | −33.0 | 0.022 |
| (total mol) | | | | |
| TG Adipose | 91.6 ± 34.6 | 72.9 ± 18.1 | −20.4 | 0.321 |
| (total mg) | (n = 5) | (n = 6) | | |
| NEFA Adipose | 18.1 ± 5.8 | 15.3 ± 2.4 | −15.8 | 0.345 |
| (total mmol) | (n = 5) | (n = 6) | | |
| TG Serum | 46.2 ± 17.0 | 47.0 ± 18.4 | 1.6 | 0.928 |
| (mg/dl) | | | | |
| NEFA Serum | 0.68 ± 0.10 | 0.64 ± 0.14 | −5.3 | 0.526 |
| (mmol/l) | | | | |
| Leptin Serum | 15.9 ± 7.2 | 10.1 ± 4.7 | −37.6 | 0.049 |
| (ng/mL) | | | | |
| Resistin Serum | 4.3 ± 1.2 | 3.6 ± 1.0 | −15.8 | 0.200 |
| (ng/mL) | | | | |
| Adiponectin Serum | 9.2 ± 1.7 | 7.0 ± 1.4 | −23.5 | <0.01 |
| (μg/mL) | | | | |
| Osteopontin Serum | 197.8 ± 22.8 | 183.0 ± 39.6 | −7.5 | 0.409 |
| (ng/mL) | | | | |
| Osteocalcin Serum | 55.7 ± 17.2 | 47.6 ± 7.8 | −14.6 | 0.218 |
| (ng/mL) | | | | |

At sacrifice, fasting serum levels of adipokines were lower in LMMS as compared to CON. Circulating levels of leptin were 35.3% (p=0.05) lower, adiponectin was 21.8% (p=0.009) lower, and resistin was 15.8% lower (p=0.26) than CON (Table 4 above). Circulating serum osteopontin (−7.5%, p=0.41) and osteocalcin (−14.6%, p=0.22) levels were not significantly affected by the mechanical signals.

### Example 11

### LMMS Fails to Reduce Existing Adiposity

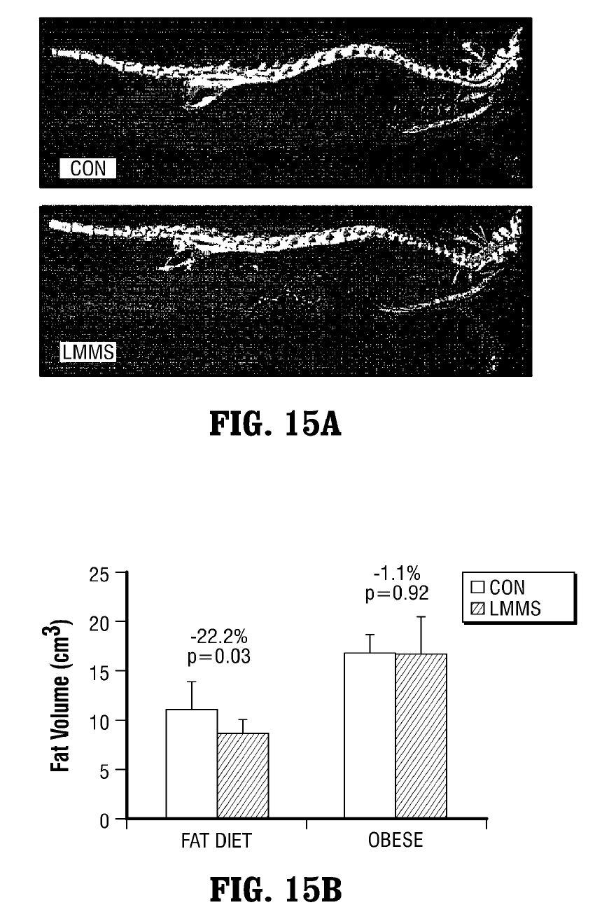

In the “reversal” model of obesity, 4 w old animals were started on a high fat diet for 3 w prior to beginning the LMMS protocol at 7 w of age. These “obese” animals were on average 3.7 grams heavier (p<0.001) than chow fed regular diet animals (baseline) at the start of the protocol. The early-adolescent obesity in these mice translated to adulthood, such that by the end of the 12 w protocol, they weighed 21% more than the CON animals who begun the fat diet at 7 w of age (p<0.001). In stark contrast to the “prevention” animals, where LMMS realized a 22.2% (p=0.03) lower overall adipose volume relative to CON (distal tibia to the base of the skull), no differences were seen for fat (−1.1%, p=0.92), lean (+1.3%, p=0.85), or bone volume (−0.2%, p=0.94) between LMMS and sham control groups after 12 w of LMMS for these already obese mice (FIG. 15).

### Example 12

### LMMS Promotes Bone and Muscle and Suppresses Visceral Fat

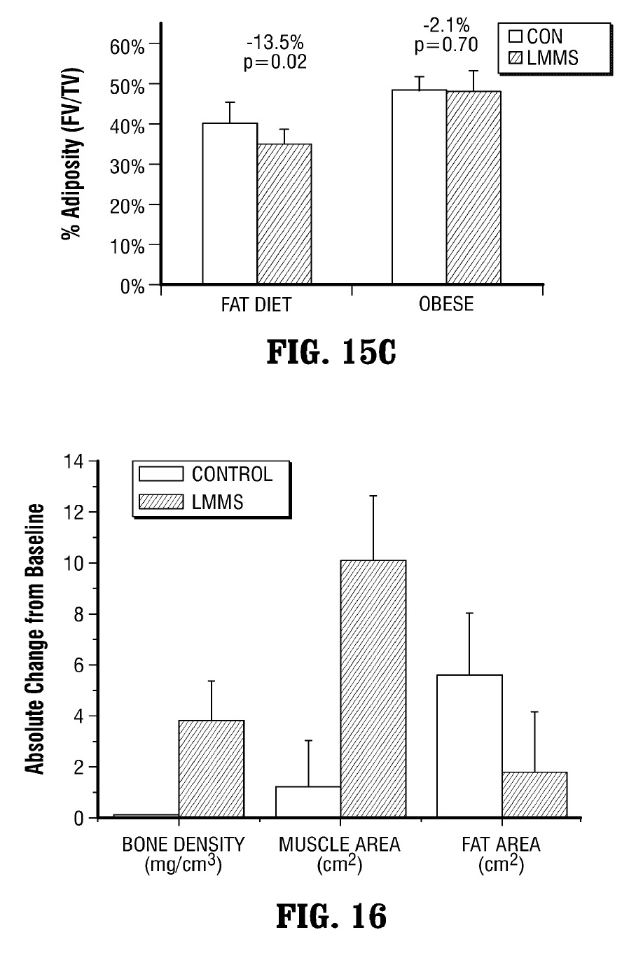

To determine whether the capacity of LMMS to suppress adiposity and increase osteogenesis in mice can translate to the human, young women with low bone density were subject to daily exposure to LMMS for 12 months. The study cohort ranged from 15-20 years old, and represented an osteopenic cohort. Detailed descriptions of this study population are provided elsewhere. 18 Over the course of one year, women (n=24) in the CON group had no significant change in cancellous bone density of the spine (0.1 mg/cm³±s.e. 1.5; FIG. 16), as compared to a 3.8 mg/cm³±1.6 increase in the spine of the LMMS treated cohort (p=0.06). Further, the average area of paraspinous muscle at the midportion of the third lumbar vertebrae, which failed to change in CON (1.2 cm²±1.9), was sharply elevated in the LMMS women (10.1 cm²+2.5; p=0.002). The area of visceral fat measured at the lumbrosacral region of CON subjects increased significantly from baseline by 5.6 cm²±2.4 (p=0.015). In contrast, the area of visceral fat measured in LMMS subjects increased by only 1.8 cm²±2.3, which was not significantly different from baseline (p=0.22). The 3.8 cm² difference in visceral fat area between groups showed a trend towards significance (p=0.13).

### Example 13

### LMMS Effects on Adipose Tissue Volume and Distribution

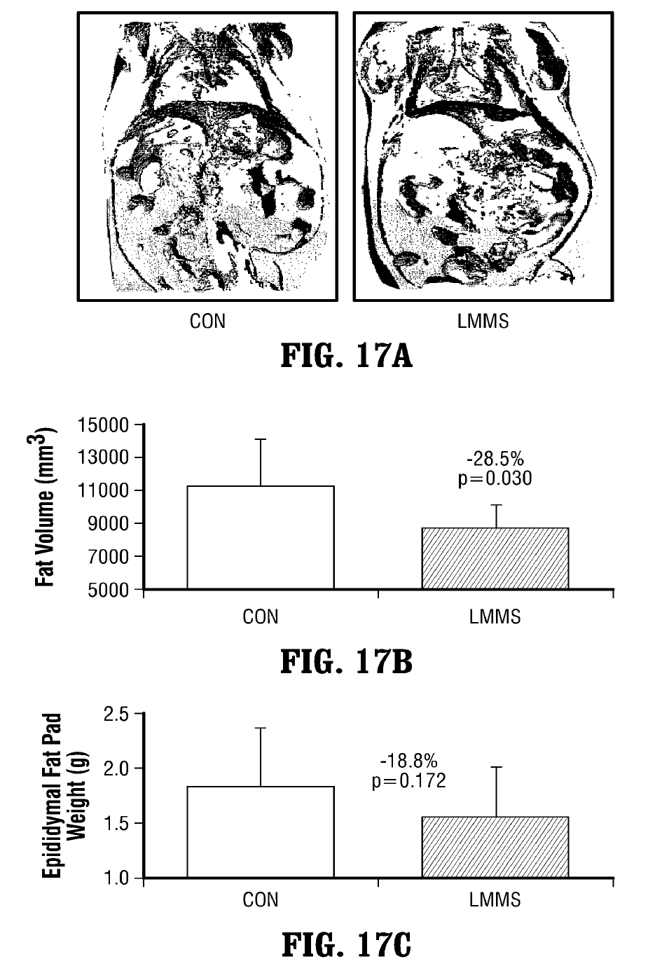

In a mouse model of dietary induced obesity, young male C57/B16 mice were fed a high fat diet where the fat content represented 45% of the calories. The LMMS stimulus (90 Hz, 0.2 g acceleration) was applied to the treatment group (n=12) for 15 min/d, 5 d/wk. A control group of animals fed the same diet but not treated with LMMS was maintained. After twelve weeks of treatment, the LMMS animals exhibited a statistically significant 28.5% reduction in total adipose volume when compared to the untreated controls, as measured by whole body vivaCT scanning. The whole body images were digitally filtered and segmented so that only fat tissue (excluding bone, organs, and muscle) would be measured. When the animals were sacrificed two weeks later, the epididymal fat pad was harvested from each animal and weighed. The decrease in fat volume based on image analysis was paralleled by a decrease of the weight of the actual epididymal fat pad harvested at sacrifice. (FIG. 17).

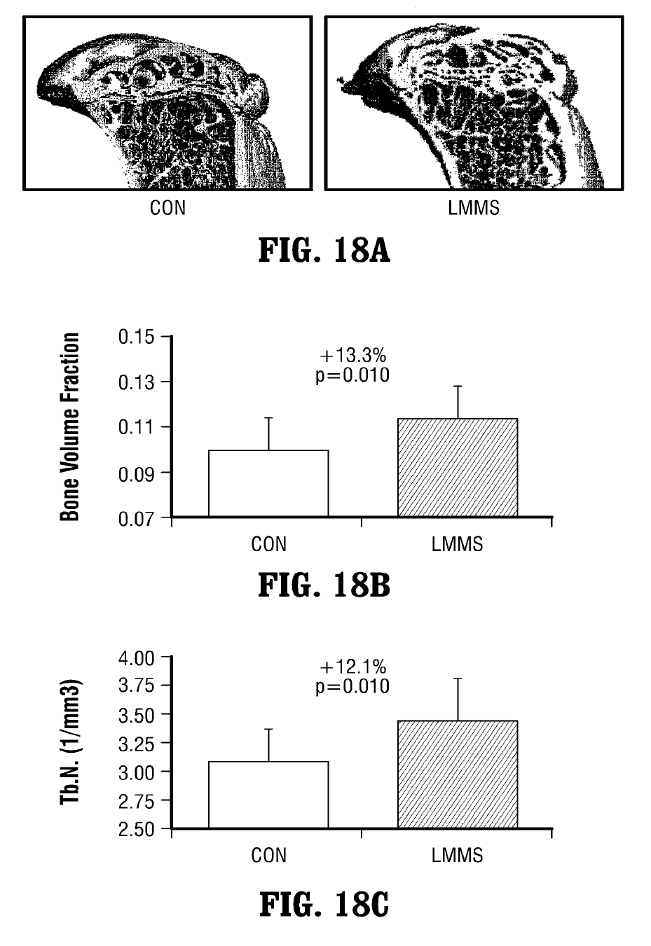

In parallel to measured decrease in fat weight and volume, these same animals exhibited an increase in their trabecular bone volume. In the proximal tibia, LMMS treated animals showed an increase in bone volume fraction of 13.3%. Microarchitectural parameters of connectivity density and trabecular number were also significantly increased, indicating better quality of bone (FIG. 18).

### Example 14

### LMMS Effects on Mesenchymal Stem Cell Numbers

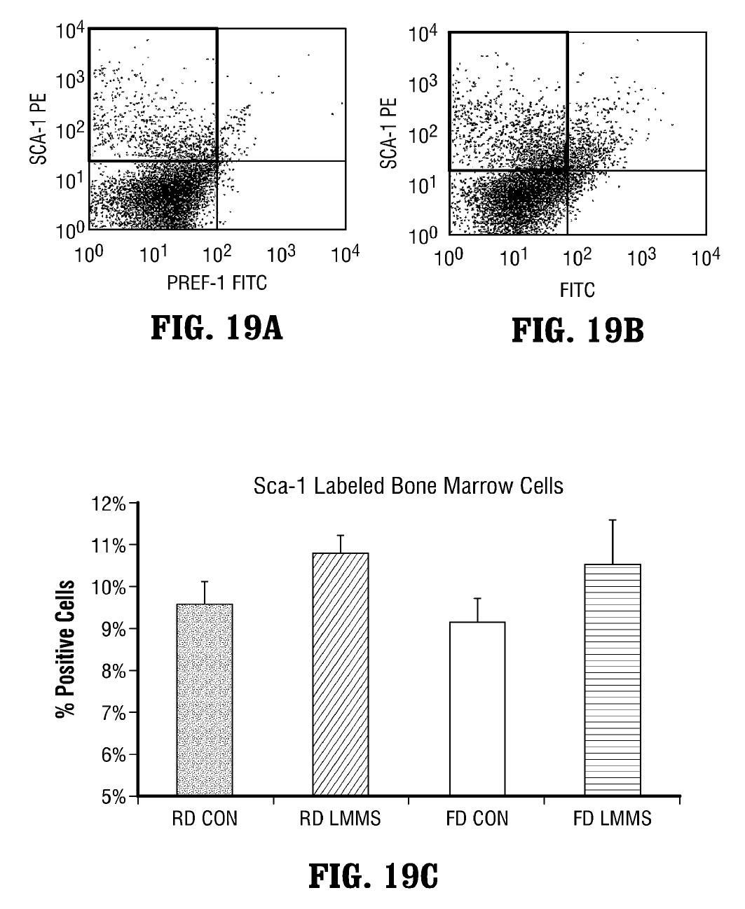

Using flow cytometry, mesenchymal stem cells can be identified out of a population of total bone marrow harvested cells by surface staining for Stem Cell Antigen-1 (Sca-1). Fluorescence conjugated anti-Sca-1 antibodies will bind only to cells expressing this surface antigen, including MSCs, allowing an accurate method to quantify stem cell number between different populations. With this method, it was demonstrated that 6 weeks of LMMS treatment applied via whole body vibration to a mouse can increase the number of MSC's by a statistically significant 19.9% (p=0.001). (FIG. 19)

### Example 15

### LMMS Effects on Stem Cell Proliferation in a Bone Marrow Transplant Model

To determine the ability of the LMMS signal to direct the differentiation pathway of stem cells, we utilized a bone marrow transplant model where GFP labeled bone marrow from a heterozygous animals was harvested and injected into sub-lethally irradiated wild-type mice. The GFP transplanted cells localize to the bone marrow cavity in the recipient mice, and repopulate the radiation damaged cells. With this model, it is possible to track the differentiation of stem cells as they retain their green fluorescence even after fully differentiating into a mature cell type. We subjected a population of bone marrow transplanted mice to 6 weeks of the LMMS treatment. At sacrifice, bone marrow, blood (after treatment to lyse the red blood cells), and adipocytes isolated by collagenase digestion from the epididymal fat pad were harvested and analysed by flow cytometry for GFP expression to track cell differentiation. Flow cytometry data utilized non-treated, age matched bone marrow transplant control animals as basal “normalization” controls.

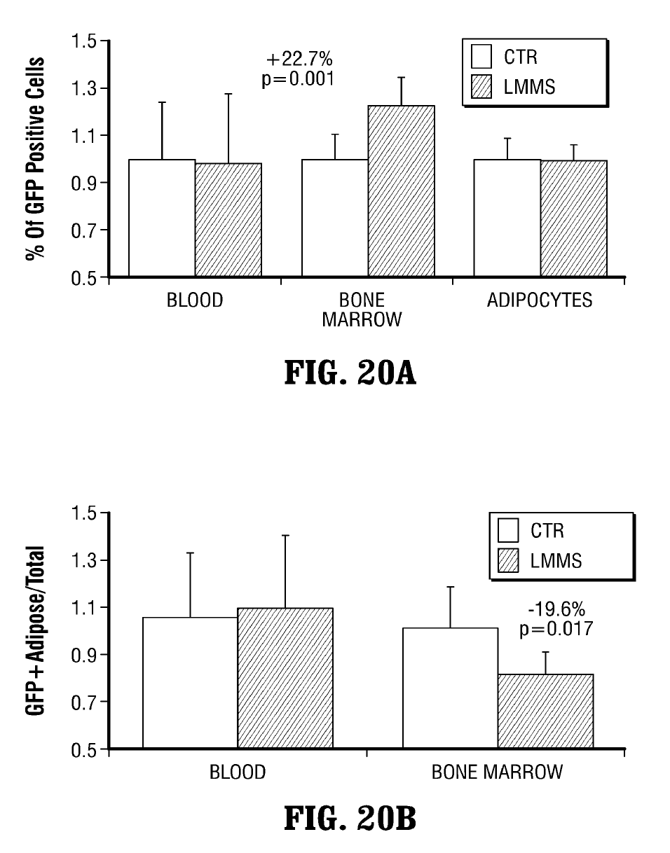

FIG. 20 summarizes data collected from the bone marrow transplant animal study. We confirm data presented in FIG. 12, that LMMS treatment increased the amount of GFP positive cells in the marrow compartment (+22.7%, p=0.001). In addition, normalized to the increased number of progenitor cells (MSCs), the number of GFP positive adipocytes was reduced by 19.6%, showing that fewer cells were differentiating into adipose tissue (FIG. 20.)

### Example 16

### Low Intensity Vibration Effects on Muscle Healing

“Normal” C57BL/6 mice, while under isoflurane anesthesia, were subjected to full thickness laceration injury through the lateral head of the gastrocnemius muscle. Care was taken to avoid injury to the neurovascular supply of the muscle. The mice were separated into either a vibrated group or a non-vibrated (control) group. Starting 8 hours after wounding, the mice of the vibrated group were subjected to daily bouts of low intensity vibration. For each bout of low intensity vibration, the mice were placed in an empty cage on a vertically vibrating platform, and low intensity vibration was applied with a peak-to-peak amplitude of 0.4 g and a frequency of 45 Hz for 30 minutes. At this amplitude (<100 μm), the vibration is barely perceptible to human touch.

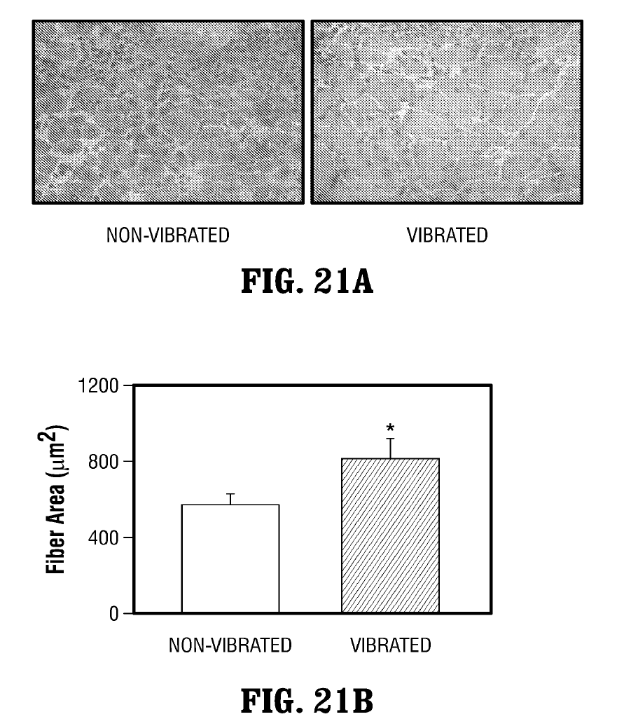

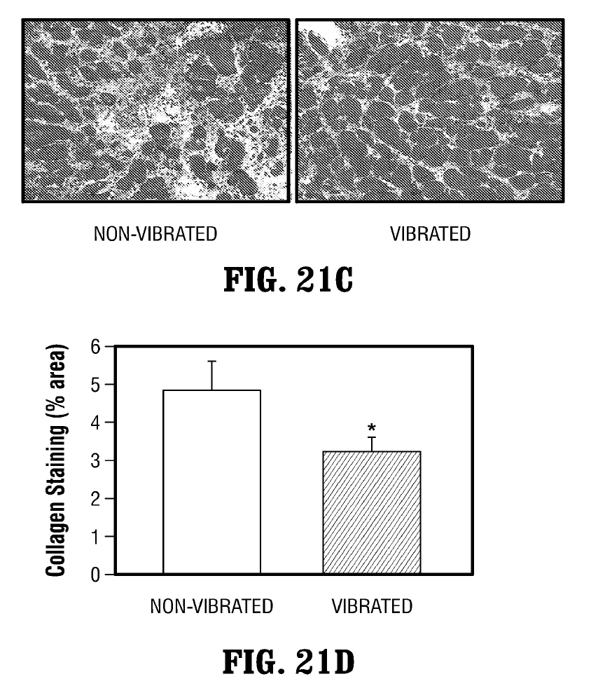

At 14 days post-injury, the muscles were harvested and healing was assessed by histological analysis. Cryosections of the gastrocnemius muscle were stained with hematoxylin and eosin for visualization of the muscle fibers, and images were captured by microscope with a 40× objective (Eclipse 80i microscope, Nikon Instruments, Inc.) (FIG. 21A.) The fibers were also measured (NIS Element Software, Nikon Instruments, Inc.) (FIG. 21B.) Fiber area was greater in muscle of the vibrated mice when compared with the controls (p<0.05; n=6 in each group). Mean and standard deviations are shown.

Cryosections of the gastrocnemius muscle were stained with Masson's Trichrome for visualization of collagen, and images were captured by microscope with a 20× objective (Nikon Instruments 80i microscope, Nikon Instruments, Inc.). (FIG. 21C.) The percentage of the area stained was measured. (NIS Elements Software, Nikon Instruments, Inc.) (FIG. 21D.) Collagen staining was greater in muscle of the control mice when compared with the vibrated mice (p<0.05; n=6 in each group). Mean and standard deviations are shown.

These data indicate that low intensity vibration enhances growth of muscle fibers and reduce fibrosis and suggest that low intensity vibration may improve healing of muscle following traumatic injury.

### Example 17

### Low Intensity Vibration Effects on Wound Healing in Diabetic Mice

8 mm diameter full-thickness wounds were created on the backs of db/db mice and covered with a Tegaderm dressing (3M Health Care) to keep the wounds moist. Diabetic db/db mice exhibit significantly impaired angiogenesis and delayed healing of excisional wounds compared to “normal” mice. The diabetic mice were separated into a vibrated group and a non-vibrated (control) group. Starting 8 hours after wounding, the mice of the vibrated group were subjected to daily bouts of low intensity vibration. For each bout of low intensity vibration, mice were placed in an empty shoebox cage on a vibrating platform, and low intensity vibration was applied with a peak-to peak amplitude of 0.4 g and a frequency of 45 Hz for 30 minutes.

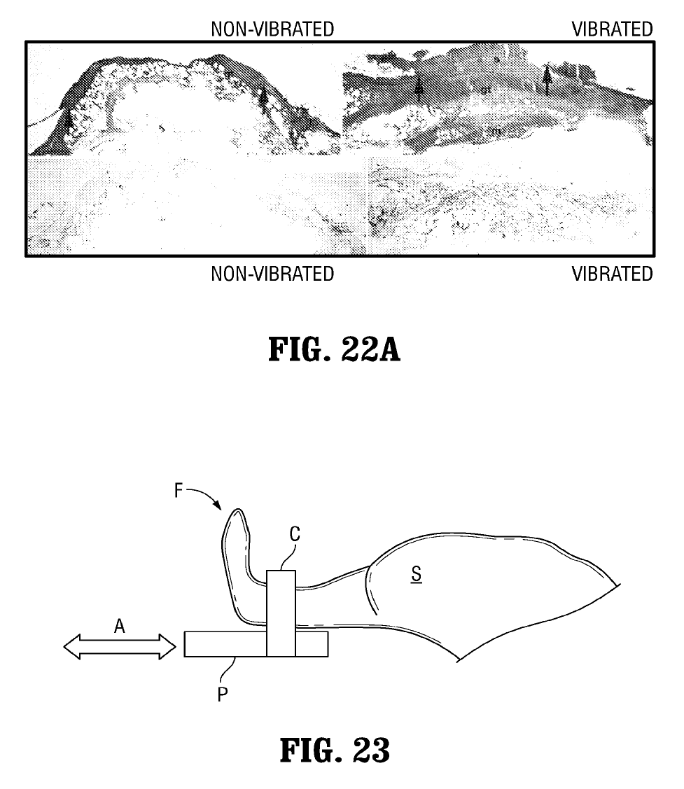

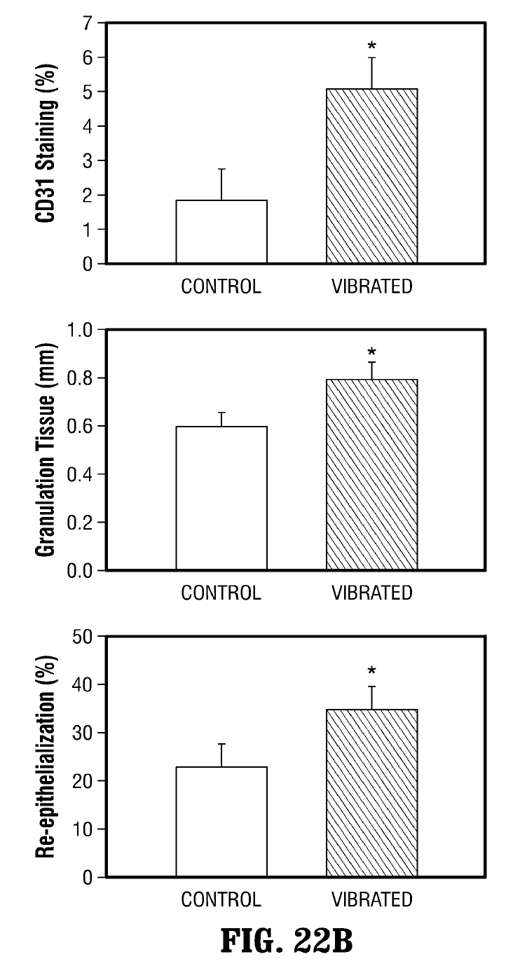

At 7 days post-injury, the tissue was harvested and healing was assessed by histological analysis. Cryosections of the wound tissue were stained with hematoxylin and eosin and images were captured. As shown in the top row of images in FIG. 22a, arrows indicate ends of epithelia tongues, “s” indicates fibrin scab, “gt” indicates granulation tissue, and “m” indicates muscle layer. Re-epithelialization and granulation tissue thickness were increased in wounds of mice subjected to vibration. Fibrin scab was present in some wounds and other times removed along with the Tegaderm dressing. The presence of scab did not appear to be affected by vibration.

Angiogeneisis was assessed by immunohistochemical staining for the endothelial cell marker CD31. (Bottom row of FIG. 22A). CD31 labeling was significantly increased in wounds of mice subjected to vibration.

Summary data for angiogenesis assessed by percentage of section that stained positive for CD31, granulation tissue thickness assessed in hematoxylin and eosin stained sections, and re-epithelialization assessed as percentage of wound length are illustrated in FIG. 22B. Angiogenesis, granulation thickness, and re-eithelialization were greater in tissue of the vibrated mice when compared with the controls (p<0.05; n=6 in each group). Mean and standard deviations are shown.

A number of embodiments of the invention have been described. Nevertheless, it will be understood that various modifications may be made without departing from the spirit and scope of the invention. Accordingly, other embodiments are within the scope of the following claims.

1. A method of reducing or suppressing pancreatitis in a subject, the method comprising administering to the subject a low magnitude, high frequency mechanical signal on a period basis and for a time sufficient to reduce or suppress pancreatitis.

2.-39. (canceled)
